SEQUENCE LISTING (52 sequences)

SEQ ID NO: 1 - DNA, 17 bp, Artificial Sequence
  CRHR1-F primer
caacacgacaaacatgg

SEQ ID NO: 2 - DNA, 18 bp, Artificial Sequence
  CRHR1-R primer
gcaagaagaggacaaagg

SEQ ID NO: 3 - DNA, 18 bp, Artificial Sequence
  CRHR2-F primer
tcatcaccaccttcatcc

SEQ ID NO: 4 - DNA, 18 bp, Artificial Sequence
  CRHR2-R primer
cagccttccacaaacatc

SEQ ID NO: 5 - DNA, 20 bp, Artificial Sequence
  UCN-F primer
cggcgaatgtggtccaggat

SEQ ID NO: 6 - DNA, 20 bp, Artificial Sequence
  UCN-R primer
ccgatcacttgcccaccgaa

SEQ ID NO: 7 - DNA, 21 bp, Artificial Sequence
  TH-F primer
ccagacctcgtctcctttgta

SEQ ID NO: 8 - DNA, 20 bp, Artificial Sequence
  TH-R primer
gggctggtgcaatcagttag

SEQ ID NO: 9 - DNA, 22 bp, Artificial Sequence
  DAT-F primer
ttgggtttggagtgctgattgc

SEQ ID NO: 10 - DNA, 21 bp, Artificial Sequence
  DAT-R primer
agaagacgacgaagccagagg

SEQ ID NO: 11 - DNA, 21 bp, Artificial Sequence
  AADC-F primer
cctactggctgctcggactaa

SEQ ID NO: 12 - DNA, 21 bp, Artificial Sequence
  AADC-R primer
gcgtaccagtgactcaaactc

SEQ ID NO: 13 - DNA, 18 bp, Artificial Sequence
  VMAT2-F primer
ctttggagttggttttgc

SEQ ID NO: 14 - DNA, 18 bp, Artificial Sequence
  VMAT2-R primer
gcagttgtggtccatgag

SEQ ID NO: 15 - DNA, 24 bp, Artificial Sequence
  GAD67-F primer
aattgcacccgtgtttgttcttat

SEQ ID NO: 16 - DNA, 21 bp, Artificial Sequence
  GAD67-R primer
agcgcagccccagccttcttt

SEQ ID NO: 17 - DNA, 20 bp, Artificial Sequence
  SERT-F primer
gacagcacgttcgcaggcct

SEQ ID NO: 18 - DNA, 20 bp, Artificial Sequence
  SERT-R primer
gacagcacgttcgcaggcct

SEQ ID NO: 19 - DNA, 23 bp, Artificial Sequence
  VGLUT1-F primer
ggcagtttccaggacctccactc

SEQ ID NO: 20 - DNA, 26 bp, Artificial Sequence
  VGLUT1-R primer
gcaagaggcagttgagaaggagagag

SEQ ID NO: 21 - DNA, 20 bp, Artificial Sequence
  Foxa2-F primer
gtatgctgggagccgtgaag

SEQ ID NO: 22 - DNA, 18 bp, Artificial Sequence
  Foxa2-R primer
agcctgcgctcatgttgc

SEQ ID NO: 23 - DNA, 20 bp, Artificial Sequence
  Lmx1a-F primer
accagcgagccaagatgaag

SEQ ID NO: 24 - DNA, 19 bp, Artificial Sequence
  Lmx1a-R primer
gccagcattgccactacca

SEQ ID NO: 25 - DNA, 24 bp, Artificial Sequence
  Lmx1b-F primer
aactgtactgcaaacaagactacc

SEQ ID NO: 26 - DNA, 23 bp, Artificial Sequence
  Lmx1b-R primer
ttcatgtccccatcttcatcctc

SEQ ID NO: 27 - DNA, 18 bp, Artificial Sequence
  Pitx3-F primer
gcaactggccgcccaagg

SEQ ID NO: 28 - DNA, 18 bp, Artificial Sequence
  Pitx3-R primer
aggccccacgttgaccga

SEQ ID NO: 29 - DNA, 21 bp, Artificial Sequence
  Nurr1-F primer
cgcgtcgcagttgcttgacac

SEQ ID NO: 30 - DNA, 21 bp, Artificial Sequence
  Nurr1-R primer
cctggaacctggaatagtcca

SEQ ID NO: 31 - DNA, 20 bp, Artificial Sequence
  EN1-F primer
cacgcaccaggaagctaaag

SEQ ID NO: 32 - DNA, 20 bp, Artificial Sequence
  EN1-R primer
ctcgtctttgtcctggaccg

SEQ ID NO: 33 - DNA, 22 bp, Artificial Sequence
  EN2-F primer
aaccgtgaacaaagggccagtg

SEQ ID NO: 34 - DNA, 20 bp, Artificial Sequence
  EN2-R primer
agaaacagccccctttgcag

SEQ ID NO: 35 - DNA, 20 bp, Artificial Sequence
  ]-actin-F primer
cacccgcgagtacaaccttc

SEQ ID NO: 36 - DNA, 20 bp, Artificial Sequence
  ]-actin-R primer
cccatacccaccatcacacc

SEQ ID NO: 37 - DNA, 20 bp, Artificial Sequence
  10CpG-F primer
ggtttggttagagagctcta

SEQ ID NO: 38 - DNA, 20 bp, Artificial Sequence
  10CpG-R primer
ccacgcctgctgtgcctgag

SEQ ID NO: 39 - DNA, 23 bp, Artificial Sequence
  NB1-F primer
agaggatgcgcaggaggtaggag

SEQ ID NO: 40 - DNA, 20 bp, Artificial Sequence
  NB1-R primer
gtcccgagttctgtctccac

SEQ ID NO: 41 - DNA, 20 bp, Artificial Sequence
  NB2-F primer
tcctggaggggactttatga

SEQ ID NO: 42 - DNA, 20 bp, Artificial Sequence
  NB2-R primer
ctggatttcctaagggctca

SEQ ID NO: 43 - DNA, 20 bp, Artificial Sequence
  NB3-F primer
gggtgtggatgctaactgga

SEQ ID NO: 44 - DNA, 20 bp, Artificial Sequence
  NB3-R primer
agtggtagccccattctcag

SEQ ID NO: 45 - RNA, 19 nt, Rattus rattus
  misc_RNA (1)..(19)
cugucauucuaaauaggga

SEQ ID NO: 46 - RNA, 19 nt, Rattus rattus
  misc_RNA (1)..(19)
ggguuguauugauguuuaa

SEQ ID NO: 47 - RNA, 19 nt, Rattus rattus
  misc_RNA (1)..(19)
gggucugauuuaauuuaug

SEQ ID NO: 48 - RNA, 19 nt, Rattus rattus
  misc_RNA (1)..(19)
ccauguaguuuuaacagaa

SEQ ID NO: 49 - RNA, 19 nt, Rattus rattus
  misc_RNA (1)..(19)
ccuucaacucggucaacgu

SEQ ID NO: 50 - RNA, 19 nt, Rattus rattus
  misc_RNA (1)..(19)
cuccuccccuuauguauac

SEQ ID NO: 51 - RNA, 19 nt, Rattus rattus
  misc_RNA (1)..(19)
gcgcugucauugucagaug

SEQ ID NO: 52 - RNA, 19 nt, Rattus rattus
  misc_RNA (1)..(19)
uucacaaccuguauucuca

(19) United States
(12) Patent Application Publication
(10) Pub. No.: US 2016/0355782 A1
(43) Pub. Date: Dec. 8, 2016

(54) METHOD FOR ENHANCING DIFFERENTIATION OF DOPAMINERGIC NEURONS

(71) Applicant: Buddhist Tzu Chi Medical Foundation, Hualien City (TW)

(72) Inventors: Mei-Jen Wang, Hualien City (TW); Hsin-Yi Huang, Hualien City (TW); Tsung-Lang Chiu, Hualien City (TW); Hui-Fen Chang, Hualien City (TW); Hui-Ru Hsu, Hualien City (TW); Cheng-Yoong Pang, Hualien City (TW); Hock-Kean Liew, Hualien City (TW)

(73) Assignee: BUDDHIST TZU CHI MEDICAL FOUNDATION, Hualien City (TW)

(21) Appl. No.: 14/732,889

(22) Filed: Jun. 8, 2015

(51) Int. Cl. C12N 5/0793

(52) U.S. Cl. CPC C12N 5/0619; C12N 2506/08; C12N 2501/998

(57) ABSTRACT

A method for enhancing differentiation of dopaminergic (DA) neurons is provided. The method of the present invention includes culturing precursor cells; administering urocortin to the precursor cells; and differentiating the precursor cells into the DA neurons.

### BACKGROUND OF THE INVENTION

1. Field of the Invention

The present invention relates to methods for enhancing differentiation of dopaminergic (DA) neurons, relates particularly to a method for enhancing differentiation of DA neurons by administering urocortin.

2. Description of Related Art

Midbrain dopaminergic (mDA) neurons play important roles in the regulation of motor performances, behavior, and cognition. During development, the induction and further specification of dopaminergic (DA) precursors as well as the differentiation of DA precursors towards mature DA neurons within the ventral midbrain (VM) involve a complex spatial and temporal cascade of coordinated transcriptional regulators (intrinsic) and diffusible signals (extrinsic) (Prakash and Wurst, 2006; Abeliovich and Hammond, 2007).

Accordingly, early proliferating neuroepithelial cells in the midbrain floor plate become committed to the mDA-inductive pathway early in development. This is initiated by extracellular gradients of fibroblast growth factor 8 (FGF8) and sonic hedgehog (Shh) from the midbrain-hindbrain boundary and the ventral neural tube, respectively (Ye et al., 1998).

In addition, Wnt1 and transforming growth factor-β probably also play a role in the patterning of midbrain progenitors (Farkas et al., 2003; Prakash et al., 2006).

These signals result in the activation of a combination of transcription factors, including Otx2, Lmx1a/b, En1/2, Msx1/2, Foxa2, Ngn2 and Mash1, in a temporal sequence (Abeliovich and Hammond, 2007; Ang, 2006). These transcription factors regulate the specification and differentiation of progenitors into mDA neurons. In the development of postmitotic mDA neuron progenitors, Nurr1, the DA key-fate determining transcription factor, Lmx1b, and Ent/2 function in parallel to induce aspects of the postmitotic mDA neurons. Furthermore, Nurr1 and Pitx3 act cooperatively to induce late markers of the mDA neurons phenotype.

In contrast to this detailed information about early inductive signals and late key fate-determining transcription factors regulating terminal differentiation, the cell-extrinsic factors inducing this terminal differentiation are less well understood.

Urocortin (hereinafter also referred to as UCN) is a peptide comprising 40 amino acids that is closely related to the neuroendocrine hypothalamic hormone and corticotropin-releasing hormone (CRH). The function of both neuropeptides is to regulate neuroendocrine, autonomic, and immunologic responses to stress (Lovejoy and Balment, 1999).

UCN is expressed in a variety of adult rat brain regions and has been shown to participate in the regulation of anxiety, learning, and memory in the brain (Oki and Sasano, 2004; Pan and Kastin, 2008). The action of UCN is mediated by binding to two distinct receptors, CRHR1 and CRHR2. Accumulated studies showed that UCN appears to protect neurons against oxidative stress-, excitotoxicity-, or aging-induced cell death (Pedersen et al., 2002; Bayatti et al., 2003; Huang et al., 2011).

DA neurons could be therapeutic in certain situations. For instance, neural transplantation is a clinically promising experimental treatment in a patient having neuron associated disorder.

However, there are few methods for enhancing differentiation of midbrain DA neurons. Accordingly, it is an urgent and important issue to provide a method to enhance differentiation of DA neurons efficiently.

### SUMMARY OF THE INVENTION

In an aspect of the present invention, a method for enhancing differentiation of dopaminergic (DA) neurons is provided. In accordance with the present invention, the method comprises culturing precursor cells; administering UCN to the precursor cells; and differentiating the precursor cells into the DA neurons.

In one embodiment of the present invention, the DA neurons are midbrain dopaminergic neurons. In another embodiment of the present invention, the cells are midbrain DA precursor cells, Nurr1-expressing neural precursor cells (NPCs), or DA neuroblastoma. Preferably, the cells are midbrain DA precursor cells.

In one embodiment of the present invention, the differentiation of DA neurons is late differentiation of DA precursor cells.

In one embodiment of the present invention, the UCN is administered at an amount of 0.25 μM to 1 μM. In a preferred embodiment of the present invention, the UCN is administered at an amount of 0.5 μM to 1 μM. Preferably, the UCN is administered at an amount of 1 μM.

In one embodiment of the present invention, the precursor cells are administered with the UCN for 1 to 8 days.

In one embodiment of the present invention, after the administration, the level of at least one DA phenotype gene in the precursor cells is increased. In some embodiments of the present invention, the DA phenotype gene is selected from the group consisting of tyrosine hydroxylase (TH), dopamine transporter (DAT), 1-aromatic amino acid decarboxylase (AADC), and vesicular monoamine transporter (VMAT2).

In one embodiment of the present invention, after the administration, the activity of histone deacetylase (HDAC) in the precursor cells is inhibited, and the level of acetylated histone H3 (Ac-H3) is increased. In another embodiment of the present invention, the level of the transcription factors, Nurr1, Foxa2, and Pitx3, in the precursor cells is increased.

In another aspect of the present invention, a method for producing midbrain DA neurons is provided. In accordance with the present invention, the method comprises administering UCN to midbrain DA precursor cells; and differentiating the midbrain DA precursor cells into the midbrain DA neurons.

### BRIEF DESCRIPTION OF THE DRAWINGS

FIGS. 1A to 1D show the co-localization of UCN and CRHRs in postmitotic mDA progenitors in vivo and in vitro. FIGS. 1A to 1C are images of immunofluorescence staining for UCN, CRHR1, CRHR2, Lmx1b, Nurr1 and TH in the ventral midbrain (VM) of E11.5 (FIG. 1A), E13.5 (FIG. 1B), and E14.5 (FIG. 1C) rat embryos. The VM of rat embryos were cryo-sectioned and subjected to immunofluorescence staining. Sections then were examined by confocal microscopy. FIG. 1D is an image of double immuno-staining of VM precursor cultures with an antibody specific for Nurr1 and antibodies for UCN, CRHR1 and CRHR2.

FIGS. 2A to 2J show that DA neuron differentiation was enhanced by UCN. FIGS. 2A and 2B show the numbers of TH⁺ neurons treated with various concentrations of UCN for 3 days (FIG. 2A) or treated with 1 μM UCN or control medium for the indicated time (FIG. 2B) (*p<0.05; **p<0.01 compared with respective control). FIG. 2C is an image of TH immuno-staining of TH-immuno-reactive cells after treatment with 1 μM UCN or control medium. FIG. 2D is a bar chart of the mRNA expression values of DA phenotype genes (*p<0.05; p<0.01 compared with respective control). FIG. 2E is an image of double immuno-staining showing co-expressions of neuron-(NeuN, MAP2) and DA neuron-specific protein (DAT) in the TH⁺ cells in the UCN-treated cultures. FIG. 2F depicts DAT-mediated specific DA uptake. For all the real-time reverse transcription polymerase chain reaction (RT-PCR), immuno-cytochemical, and functional analyses, cultures were differentiated with or without 1 μM UCN for 4 days (*p<0.05: **p<0.01 compared with respective control). FIG. 2G is a bar chart showing the differentiation phenotypes of VM precursors by the mRNA expression values of DAergic (Th), glutamatergic (Vglut1), GABAergic (Gad67), serotonergic (Sert) neuron-specific markers treated with UCN or control medium (p<0.01 compared with respective control). FIG. 2H depicts the number of TH⁺ neurons in VM precursor cultures after treatment with 15 μg/ml IgG, anti-UCN antibody, or control medium (**p<0.01 compared with IgG-treated group). FIGS. 2I and 2J show an image of immuno-blot of TH and β-Actin proteins in the cell extracts prepared from SH-SY5Y cells after treatment with UCN or control medium and the ratio of band intensity of TH and β-Actin, respectively (**p<0.01 compared with control). Con: control medium; SH-SY5Y cells: human DA neuro-blastoma.

FIGS. 3A to 3K show that UCN promotes dopaminergic (DA) neuron precursors to acquire DA phenotype. FIGS. 3A and 3B show an image of double immuno-staining of Nurr1 and TH for VM precursor cultures after treatment with UCN or control medium (FIG. 3A) and the ratio of TH⁺ to Nurr1⁺ cells (FIG. 3B), respectively (**p<0.01 compared with respective control). FIGS. 3C and 3E show that UCN enhances TH⁺ cell differentiation induced by exogenous Nurr1 in non-DA neural precursor cells (NPCs). NPCs derived from rat embryonic cortices at E13.5 were transduced with GFP or Nurr1-expressing lentiviruses, and then differentiated for 3 days in the absence or presence of 0.5 μM UCN. FIGS. 3C and 3E are an image of TH immuno-staining and an image of double immuno-staining of GFP and Nurr1 in NPCs, and FIGS. 3D and 3F show the numbers of TH⁺ neurons and the ratio of TH⁺ to Nurr1⁺ cells (*p<0.05; **p<0.01 compared with untreated Nurr1-transduced cells). FIGS. 3G to 3I show the mRNA expression values of DA phenotype genes (FIG. 3D), double immuno-staining of DAT and TH (FIG. 3E), and DAT mediated specific DA uptake (FIG. 3F) in Nurr1-transduced cortical NPCs after treatment with 0.5 μM UCN or control medium for 3 days (FIG. 3D) or 4 days (FIGS. 4E and 4F) (*p<0.05; **p<0.01 compared with respective control). FIGS. 3J and 3K show relative luciferase activity in SH-SY5Y cells transfected with pTH6.0-GL3 alone (FIG. 3J) or cortical NPCs co-transfected with pAAV-Nurr1 and pTH6.0-GL3 (FIG. 3K) that incubated with or without 1 μM UCN for 24 hours (**p<0.01 compared with respective control). Con: control medium; GFP: green fluorescent protein; SH-SY5Y cells: human DA neuro-blastoma.

FIGS. 4A to 4J show that histone deacetylases (HDACs) inhibition contributes to UCN-caused dopaminergic neuron differentiation. FIG. 4A shows UCN induced accumulation of hyperacetylated histone H3 (Ac-H3) analyzed by Western blot analysis (Ac-H3 in VM precursor cultures exposed to 1 μM UCN for the indicated times). FIGS. 4B and 4C are images of immuno-detection of Ac-H3 in Nurr1-expressing cells (i.e., VM precursor cultures, which were treated with 1 μM UCN for 1 hour or with anti-UCN antibody for 3 days, then fixed, and double immuno-stained using anti-Nurr1 antibody and anti-Ac-H3 antibody). FIGS. 4D and 4E show that sodium butyrate (SB) mimics the effect of UCN to promote DA neuron differentiation in VM precursor cultures, which were incubated with 1.25 mM SB for 3 days, by counting the number of TH⁺ cells (FIG. 4D) and % TH⁺ cells out of total Nurr1⁺ cells (FIG. 4E) (*p<0.05; **p<0.01 compared with control). FIG. 4F shows differentiation phenotypes of VM precursors treated with 1.25 mM SB for 3 days which were determined by real-time RT-PCR analysis (**p<0.01 compared with respective control). FIG. 4G shows enhanced TH⁺ cells differentiation by SB in Nurr1-expressing cortical neural precursor cells (NPCs) transduced with Nurr1 or GFP followed by the treatment of SB for the subsequent 3 days (*p<0.05 compared with respective control). FIGS. 4H and 4I show that SB elicits TH promoter activity of SH-SY5Y cells (FIG. 4H) or cortical NPCs (FIG. 4I) which were transfected with pTH6.0-GL3 alone or pAAV-Nurr1/and pTH6.0-GL3, respectively, then were treated with 1.25 mM or 0.5 mM SB, respectively, and subjected to luciferase activity after 24 hours (*p<0.05; **p<0.01 compared with respective control). FIG. 4J shows that overexpression of HDAC1 attenuates UCN-induced TH promoter activity of SH-SY5Y cells which were co-transfected with pK7-HDAC1 and pTH6.0-GL3 for 24 hours followed by the exposure of 1 μM UCN or 1.25 mM SB for another 24 hours (**p<0.01 compared with respective mock). Con: control medium; GFP: green fluorescent protein; SB: sodium butyrate; HDAC1: histone deacetylase 1.

FIGS. 5A to 5M show that UCN facilitates Nurr1 recruitment to the TH promoter by releasing MeCP2-CoREST-HDAC1 repressor complex from the promoter. FIG. 5A depicts a schema of rat TH promoter with predicted Nurr1 (NB1-NB3) and 10 repeated CpGs consensus binding sites within −1 kb of the transcription start site (TSS). FIG. 5B depicts Chromatin immuno-precipitation (ChIP) analysis to determine Nurr1 protein enrichment at the TH promoter, wherein VM precursor cultures were treated with 1 μM UCN for 2 days (**p<0.01 compared with respective control), FIG. 5C is an image of immuno-blot of Nurr1 complexes with endogenous MeCP2, CoREST and HDAC1 proteins in the cell extracts prepared from VM precursor cells after immuno-precipitation (IP) with anti-Nurr1 antibody (or IgG as the negative control) followed by immuno-blot analyses with anti-MeCP2, anti-CoREST, and anti-HDAC1 antibodies. FIGS. 5D to 5G show ChIP analysis to determine MeCP2 (FIG. 5D), CoREST (FIG. 5E), HDAC1 (FIG. 5F) and Ac-H3 (FIG. 5G) enrichment at the TH promoter (*p<0.05; **p<0.01 compared with respective control). FIGS. 5H and 5I show that SB mimics the effect of UCN on histone H3 acetylation (FIG. 5H) and Nurr1 enrichment (FIG. 5I) at the TH promoter in VM precursor cultures by ChIP analyses (*p<0.05; **p<0.01 compared with respective control). FIGS. 5J and 5K show relative luciferase activity in SH-SY5Y cells which were co-transfected with pCMV6-Entry-Mecp2/or pCMV6-Entry-Rcor1 and pTH6.0-GL3 for 24 hours followed by the exposure of 1 μM UCN (FIG. 5J) or 1.25 mM SB (FIG. 5K) for another 24 hours (**p<0.01 compared with respective mock). FIGS. 5L and 5M depict that neutralizing UCN antibody reduces endogenous Nurr1 binding (FIG. 5M) and histone H3 acetylation (FIG. 5L) on the TH promoter in VM precursor cultures which were treated with 15 μg/ml IgG or anti-UCN antibody for 2 days and then subjected to ChIP analyses (*p<0.05; **p<0.01 compared with IgG-treated group). Con: control medium; SB: sodium butyrate; MeCP2: methyl CpG binding protein 2; CoREST: element-1 silencing transcription factor corepressor; HDAC1: histone deacetylase 1; Ac-H3: acetylated histone H3.

FIGS. 6A to 6K show that UCN-upregulated Foxa2 and Pitx3 trigger dopaminergic (DA) neuron differentiation. FIG. 6A depicts real-time RT-PCR analysis of late DA regulator expression in VM precursor cells of Nurr1, Foxa2, and Pitx3 genes after UCN treatment (*p<0.05 compared with respective control). FIGS. 6B and 6C are images of immuno-cytochemistry analysis of VM precursor cells following the exposure of 1 μM UCN for 2 to 3 days. FIG. 6D shows relative mRNA expression of Nurr1, Foxa2, and Pitx3 genes in VM precursor cells after treatment with anti-UCN antibody (or IgG as the negative control) (*p<0.05 compared with IgG-treated group). FIGS. 6E and 6F show a graph of IP assays in which physical interactions between Nurr1 and Foxa2/or Pitx3 in VM precursor cells after treatment with UCN or control medium (*p<0.05 compared with respective control). FIG. 6G depicts ChIP analysis of Nurr1, Foxa2 and Pitx3 enrichments at the Nurr1 binding sites of TH promoter. All enrichments are shown relative to IgG immuno-precipitation (*p<0.05; **p<0.01 compared with IgG). FIGS. 6H and 61 are the results of ChIP analysis of Foxa2 (FIG. 6H) and Pitx3 (FIG. 6I) enrichment binding in the TH promoter region of Nurr1 binding sites in VM precursor cells after treatment with UCN or control medium (*p<0.05; **p<0.01 compared with respective control). FIGS. 6J and 6K show the luciferase activity and the number of TH⁺ neurons of VM precursor cultures after transfection with control, Foxa2 and Pitx3 siRNA for 24 hours, and treatment with UCN (*p<0.05; **p<0.01 compared with untreated-control, siRNA transfected cells; *p<0.05; ^{##}p<0.01 compared with UCN-stimulated control siRNA transfected cells). Con: control medium.

FIGS. 7A to 7I show that UCN improves differentiation of Nurr1⁺ DA precursors in vivo. The pregnant rats on day 12.5 of gestation were intra-peritoneally injected with saline or 10 μg/kg UCN every 24 hours for 3 days (FIGS. 7H & 7I) or 4 days (FIGS. 7A, 7B, & 7E-7G) before sacrifice. FIG. 7A is an image of immuno-staining against TH of rat E16.5 mesencephalon. FIG. 7B is cell counting analysis of TH⁺ cells in the VM of E16.5 saline and UCN-treated embryos (**p<0.01 compared with saline group). FIG. 7C shows an image of immunofluorescence staining for TH in the sections of midbrain explants which were pretreated with CRHRs reference antagonist (control peptide), astressin (Ast), or CP-376395 (CP) plus astressin 2B (Ast 2B) for 30 minutes followed by treatment with or without UCN; and FIG. 7D shows the number of TH⁺ cells in each group of the VM (*p<0.05; **p<0.01 compared with untreated-control cells; ^{##}p<0.01 compared with UCN-stimulated control cells). FIG. 7E shows images of immuno-histochenistry analysis of VM in E16.5 saline or UCN-received embryos via Nurr1 and TH antibody, and FIGS. FIGS. 7F and 7G show the number of TH⁺ and Nurr1⁺ cells and the proportion of TH⁺/Nurr1⁺ cells in the VM, respectively (*p<0.05; **p<0.01 compared with saline groups). FIGS. 7H and 7I show histone H3 acetylation (H) and Nurr1 recruitments (I) at the TH promoter by ChIP analyses which were performed with chromatin prepared from E15.5 VM tissue of saline or UCN-treated embryos (*p<0.05; **p<0.01 compared with saline groups). Con: control medium.

### DETAILED DESCRIPTION OF THE INVENTION

The following specific examples are used to exemplify the present invention. A person of ordinary skills in the art can conceive the other advantages of the present invention, based on the disclosure of the specification of the present invention. The present invention can also be implemented or applied as described in different specific examples. It is possible to modify and or alter the examples for carrying out this invention without contravening its spirit and scope, for different aspects and applications.

The present invention provides a method for enhancing differentiation of DA neurons comprising culturing precursor cells; and administering UCN to the precursor cell; and differentiating the precursor cells into the DA neurons.

According to the present invention, the differentiation of DA neurons is late differentiation of DA precursor cells.

Herein, the term “late differentiation of DA neurons” or “late development of DA neurons” refer to differentiation/development of DA precursor cells in or drawn from a embryo in late developmental stage, such as embryonic day (E)12.5 to E14.5 for rat, E10.5 to E12.5 for mouse, at which post-mitotic DA precursor cells are differentiating toward mature DA neurons. Several characterized transcription factors, including Nurr1, Pitx3, En, Lmx1a/b, and Foxa2, are involved in the maturation of post-mitotic DA neurons, i.e., the late development of DA neurons. In the development of post-mitotic mDA neuron progenitors, these transcription factors function in parallel to induce aspects of the post-mitotic mDA neurons.

According to the method of the present invention, the UCN is administered at an amount of 0.25 μM to 1 μM, preferably 0.5 μM to 1 μM, more preferably 1 μM.

According to the method of the present invention, the step of administering or differentiating comprises culturing the precursor cells with the UCN for an amount of time sufficient to allow for the differentiation of the precursor cells into DA neurons. In one preferred embodiment of the present invention, the time for culturing the precursor cells with the UCN can be any value selected from a range of 1 to 8 days.

According to the method of the present invention, the DA neurons express at least one DA phenotype gene. The DA phenotype genes can be, but is not limited to, tyrosine hydroxylase (TH), dopamine transporter (DAT), 1-aromatic amino acid decarboxylase (AADC) and vesicular monoamine transporter (VMAT2). According to the method of the present invention, after the administration, the level of at least one DA phenotype gene in the precursor cells is increased.

According to the method of the present invention, the step of differentiating comprises inhibiting the activity of histone deacetylase (HDAC) and increasing the level of acetylated histone H3 (Ac-H3) in the precursor cells. The step of differentiating further comprises releasing methyl CpG binding protein 2-CoREST-HDAC1 (MeCP2-CoREST-HDAC1) repressor complex from the TH promoter, and ultimately leading to an increase in Nurr1/coactivators-mediated transcription of TH gene.

According to the method of the present invention, after the administration, the level of the transcription factors in the precursor cells is increased. The transcription factors should be Nurr1, Foxa2, and Pitx3.

In addition, the present invention further provides a method for producing midbrain DA neurons, comprising administering UCN to midbrain DA precursor cells; and differentiating the midbrain DA precursor cells into the midbrain DA neurons.

Moreover, the present invention provides a method for enhancing development of DA neurons in a subject having DA precursor cells, comprising administering an effective amount of UCN to the subject. Preferably, the subject is a mammal; more preferably, the subject is a mammalian embryo. According to the method of the present invention, the subject is a rat or a human.

Many examples have been used to illustrate the present invention. The examples below should not be taken as a limit to the scope of the invention.

### Examples

### Cell Cultures

Primary rat VM precursor cells were prepared using a well-known method in the art, for example as the protocol described by Castelo-Branco et al. (2003) and performed with some modifications.

Briefly, ventral mesencephalic tissues were dissected from E14.5 Sprague-Dawley (SD) rats, and then dissociated enzymatically (0.1% trypsin) and mechanically. Cells were seeded into 24-well (1×10⁵/cm²) culture plates pre-coated with 20 μg/mL poly-D-lysine (Sigma-Aldrich), and were maintained in 0.5 mL/well of minimum essential medium (MEM) supplemented with 10% fetal bovine serum (FBS), 10% horse serum (HS), 1 g/L glucose, 2 mM L-glutamine, 1 mM sodium pyruvate, 100 μM nonessential amino acids, 100 U/mL penicillin, and 100 μg/mL streptomycin (all purchased from Invitrogen). Cultures were maintained at 37° C. in a humidified atmosphere of 5% CO₂ and 95% air. For DA neuron differentiation, VM precursor cultures, four hours after seeding, were switched to Dulbecco's Modified Eagle's Medium (DMEM)/Ham's F12 (1:1) containing N2 supplement (Invitrogen, Carlsbad, Calif.), penicillin and streptomycin. Immediately following the switch to DMEM/F12 medium, VM precursor cells were treated with various concentration of rat UCN (from Sigma-Aldrich, St. Louis, Mo.) for the indicated periods. For neutralization of endogenous UCN bioactivity, anti-UCN antibody (15 μg/mL) (Santa Cruz Biotechnology, Santa Cruz, Calif.) was added to the VM precursor cultures.

Cultures of neural precursor cells (NPCs) derived from cortices at E13.5 were performed as the methods known by a person skilled in the art, for example, as the protocol described by Huang et al. (2012) and carried out with some modifications. Briefly, dissected cortical tissues were mechanically triturated and plated at 5×10⁴ cells on coverslips (12-mm diameter) pre-coated with 20 μg/mL fibronectin (Invitrogen)/15 μg/mL poly-L-ornithine (Sigma-Aldrich) in 24 well plates. Cells were cultured in a serum-free NPCs culture medium containing DMEMF12, 2% B27 supplement (Invitrogen), 0.6% glucose, 20 ng/mL bFGF (Sigma-Aldrich), 20 ng/mL EGF (Invitrogen), 2 μg/mL heparin (Sigma-Aldrich), 100 U/mL penicillin, and 100 μg/mL streptomycin.

TH promoter assay or lenti-virial transductions were carried out at 60%˜70% cell confluences (usually for 3 days) as described below. Cell differentiation was induced for 3 to 5 days by switching to mitogens-free DMEM/F12/N2 medium in the presence of 0.5 μM UCN. Differentiation medium was changed every other day.

Human DA neuroblastoma SH-SY5Y cells were grown in Minimal Essential Medium (MEM)/F12 medium containing 10% FBS, 2 mM L-glutamine, 100 μM nonessential amino acids, 100 U/mL penicillin, and 100 μg/mL streptomycin. Cells were incubated at 37° C. under saturating humidity in 5% CO₂/95% air.

### Midbrain Explant Cultures

Midbrain explants were dissected from E13.5 embryos by the method known by a person skilled in the art, for example as the protocol described by Baizabal and Covarrubias (2009) and performed with some modifications.

The dorsal midline of the explants was cut and the tissue was placed on porous (0.4 μm) transparent membrane inserts (25-mm in diameter, Nunc, Roskilde, Denmark) with the ventricular surface facing up. Inserts were then placed into six-well culture plates. Each well contained 1.5 mL of MEM medium supplemented with 10% FBS, 100 U/mL penicillin, and 100 μg/mL streptomycin. Explants were incubated at 37° C. with 5% CO₂ for 4 hours, after which the explants were incubated in a serum-free DMEM/F12/N2 medium with or without UCN for 3 days. Midbrain explants were then fixed with 4% paraformaldehyde and immuno-histochemical analysis was performed.

### Lenti-Viral Transduction

The lenti-viral vector expressing mouse Nurr1 was originally constructed by Malin Parmar (Pfisterer et al., 2011) and purchased from Addgene (Addgene plasmid 35000).

Production of the virus was performed using a protocol known by a person skilled in the art, for example, as the protocol described by Liu et al. (2012) and carried out with some modifications. Briefly, a plasmid mixture containing 16 μg pCMV-ΔR8.91 (packaging construct), 0.7 μg pMD.G (envelope plasmid) and 7 μg of Lenti-vectors expressing Nurr1 or green fluorescent protein (GFP) was suspended in 500 μL CaCl₂ (250 mM) and added volume for volume into 2×HBS buffer. The DNA-CaCl₂ precipitate was added to human kidney 293T cells cultured in a 75 T flask and allowed to incubate for 12-16 h before switching to fresh culture medium. The supernatant was collected 60 h after transfection, filtered through the 0.45 μm filter flask and centrifuged at 8,000×g for 12 h. The resulting pellet was re-suspended in 200 μL of DMEM, aliquoted, and stored at −80° C. The titers of the vectors used in this study were in the range of 8×10⁸ to 3×10⁹ transducing units per milliliter, which were titrated by quantitative PCR analysis (Lenti-XTM qRT-PCR Titration kit, Clontech, Mountain View, Calif.).

For lenti-viral transduction, E13 cortical NPCs were incubated with lenti-viruses (25 multiplicity of infection) expressing Nurr1 or GFP containing poly-brene (hexadimethrine bromide, 1 μg/mL, Sigma-Aldrich) for 24 hours. After 1 day of further cell expansion in the presence of the mitogens, cells were induced to differentiate.

### Real-Time RT-PCR Analysis

Total RNA was extracted from VM tissues or cultured VM precursors with TRIzol® reagent (Invitrogen). One-step real-time RT-PCR analysis was performed to determine the expression of genes (Power SYBR® Green RNA-to-C_{T}™ 1-step kit, Applied Biosystems, Foster City, Calif.). Threshold cycle (CO value for each test gene was normalized to the Cₜ value for the β-actin control from the same RNA preparations. The ratio of transcription of each gene was calculated as 2^{-(ΔCt)}, where ΔCₜ is the difference C_{r (test gene)}−C_{t (β-actin)}. The primers used herein are listed in Table 1.

TABLE 1
Real-Time PCR primers

| | | Annealing |
|---|---|---|
| Gene | Sequence (5′→3′) | temp (° C.) |
| CRHR1 | F: CAA CAC GAC AAA CA | 50 |
| | TGG | |
| | (SEQ ID NO: 1) | |
| | R: GCA AGA AGA GGA CAA | |
| | AGG | |
| | (SEQ ID NO: 2) | |
| CRHR2 | F: TCA TCA CCA CCT TCA | 50 |
| | TCC | |
| | (SEQ ID NO: 3) | |
| | R: CAG CCT TCC ACA AAC | |
| | ATC | |
| | (SEQ ID NO: 4) | |
| UCN | F: CGG CGA ATG TGG TCC | 62 |
| | AGG AT | |
| | (SEQ ID NO: 5) | |
| | R: CCG ATC ACT TGC CCA | |
| | CCG AA | |
| | (SEQ ID NO: 6) | |
| TH | F: CCA GAC CTC GTC TCC | 60 |
| | TTT GTA | |
| | (SEQ ID NO: 7) | |
| | R: GGG CTG GTG CAA TCA | |
| | GTT AG | |
| | (SEQ ID NO: 8) | |
| DAT | F: TTG GGT TTG GAG TGC | 60 |
| | TGA TTG C | |
| | (SEQ ID NO: 9) | |
| | R: AGA AGA CGA CGA AGC | |
| | CAG AGG | |
| | (SEQ ID NO: 10) | |
| AADC | F: CCT ACT GGC TGC TCG | 57 |
| | GAC TAA | |
| | (SEQ ID NO: 11) | |
| | R: GCG TAC CAG TGA CTC | |
| | AAA CTC | |
| | (SEQ ID NO: 12) | |
| VMAT2 | F: CTT TGG AGT TGG TTT | 57 |
| | TGC | |
| | (SEQ ID NO: 13) | |
| | R: GCA GTT GTG GTC CAT | |
| | GAG | |
| | (SEQ ID NO: 14) | |
| GAD67 | F: AAT TGC ACC CGT GTT | 57 |
| | TGT TCT TAT | |
| | (SEQ ID NO: 15) | |
| | R: AGC GCA GCC CCA GCC | |
| | TTC TTT | |
| | (SEQ ID NO: 16) | |
| SERT | F: GAC AGC ACG TTC GCA | 57 |
| | GGC CT | |
| | (SEQ ID NO: 17) | |
| | R: GAC AGC ACG TTC GCA | |
| | GGC CT | |
| | (SEQ ID NO: 18) | |
| VGLUT1 | F: GGC AGT TTC CAG GAC | 60 |
| | CTC CAC TC | |
| | (SEQ ID NO: 19) | |
| | R: GCA AGA GGC AG TTG | |
| | AGA AGG AGA GAG | |
| | (SEQ ID NO: 20) | |
| Foxa2 | F: GTA TGC TGG GAG CCG | 50 |
| | TGA AG | |
| | (SEQ ID NO: 21) | |
| | R: AGC CTG CGC TCA TGT | |
| | TGC | |
| | (SEQ ID NO: 22) | |
| Lmx1a | F: ACC AGC GAG CCA AGA | 50 |
| | TGA AG | |
| | (SEQ ID NO: 23) | |
| | R: GCC AGC ATT GCC ACT | |
| | ACC A | |
| | (SEQ ID NO: 24) | |
| Lmx1b | F: AAC TGT ACT GCA AAC | 55 |
| | AAG ACT ACC | |
| | (SEQ ID NO: 25) | |
| | R: TTC ATG TCC CCA TCT | |
| | TCA TCC TC | |
| | (SEQ ID NO: 26) | |
| Pitx3 | F: GCA ACT GGC CGC CCA | 57 |
| | AGG | |
| | (SEQ ID NO: 27) | |
| | R: AGG CCC CAC GTT GAC | |
| | CGA | |
| | (SEQ ID NO: 28) | |
| Nurr1 | F: CGC GTC GCA GTT GCT | 57 |
| | TGA CAC | |
| | (SEQ ID NO: 29) | |
| | R: CCT GGA ACC TGG AAT | |
| | AGT CCA | |
| | (SEQ ID NO: 30) | |
| EN1 | F: CAC GCA CCA GGA AGC | 51 |
| | TAA AG | |
| | (SEQ ID NO: 31) | |
| | R: CTC GTC TTT GTC CTG | |
| | GAC CG | |
| | (SEQ ID NO: 32) | |
| EN2 | F: AAC CGT GAA CAA AGG | 51 |
| | GCC AGT G | |
| | (SEQ ID NO: 33) | |
| | R: AGA AAC AGC CCC CTT | |
| | TGC AG | |
| | (SEQ ID NO: 34) | |
| β-actin | F: CAC CCG CGA GTA CAA | 60 |
| | CCT TC | |
| | (SEQ ID NO: 35) | |
| | R: CCC ATA CCC ACC ATC | |
| | ACA CC | |
| | (SEQ ID NO: 36) | |

### Chromatin Immuno-Precipitation (ChIP)

VM precursor cultures were treated with 1 μM UCN for about 1 to 2 days. VM cells for ChIP from rat embryos, the pregnant rats on day 12.5 of gestation were intra-peritoneally injected with saline as control or 10 μg/kg UCN as treatment every 24 h for 3 days before sacrifice. The ventral mesencephalon from E15.5 embryos was dissected. Cells were cross-linked with 1% formaldehyde, and stored at −80° C. before use.

Chromatin immuno-precipitation (ChIP) assays were performed using SimpleChIP® Enzymatic Chromatin IP kit (Cell Signaling Technology, Beverly, Mass.) by the instruction of the kit. Briefly, cross-linked chromatin in the cells was enzymatic digested to generate fragments with a length of approximately 150-900 bp (1 to 5 nucleosomes). The chromatin was subjected to immuno-precipitation using the following antibodies: anti-Nurr1 (Santa Cruz), anti-acetyl histone H3 (Ac-H3), anti-HDAC1, anti-CoREST (all from Upstate Biotechnology), anti-MeCP2 (Chemicon,), anti-Foxa2 (Cell signaling Technology), anti-Pitx3, and IgG (both from Abcam). Immuno-precipitated DNA fragments were collected by protein G magnetic beads. DNA/protein complexes were eluted from the beads and reverse cross-linked at 65° C. for 2 h in the presence of proteinase K. Purified DNA were subjected to real-time PCR using primers specific to rat TH promoter loci (referring to Table 2). The abundance of the immuno-precipitated DNA in a sample was normalized to the amount of signal in the input DNA. The values of the control samples were set to 1.0.

TABLE 2
Real-Time PCR primers
for rat TH promoter loci

| | | | Annealing |
|---|---|---|---|
| Loci | CpG | Sequence (5′→3′) | Temp (° C.) |
| 10CpG | 10 | F: GGT TTG GTT | 57 |
| | | AGA GAG CTC TA | |
| | | (SEQ ID NO: 37) | |
| | | R: CCA CGC CTG | |
| | | CTG TGC CTG AG | |
| | | (SEQ ID NO: 38) | |
| NB1 | 4 | F: AGA GGA TG CGC | 57 |
| | | AGG AGG TAG GAG | |
| | | (SEQ ID NO: 39) | |
| | | R: GTC CCG AGT | |
| | | TCTG TCT CCA C | |
| | | (SEQ ID NO: 40) | |
| NB2 | 0 | F: TCC TGG AGG | 57 |
| | | GGA CTT TAT GA | |
| | | (SEQ ID NO: 41) | |
| | | R: CTG GAT TTC | |
| | | CTA AGG GCT CA | |
| | | (SEQ ID NO: 42) | |
| NB3 | 1 | F: GGG TGT GGA | 57 |
| | | TGC TAA CTG GA | |
| | | (SEQ ID NO: 43) | |
| | | R: AGT GGT AGC | |
| | | CCC ATT CTC AG | |
| | | (SEQ ID NO: 44) | |

### Immuno-Cytochemistry

For immuno-cytochemistry, cells were fixed with 4% paraformaldehyde followed by blocking with PBS containing 0.4% Triton X-100, 3% normal goat serum and 2% bovine serum albumin (BSA) for 1 h at room temperature. After blocking, cells were incubated overnight at room temperature with primary antibodies. Primary antibodies were rabbit anti-UCN (1:200), mouse anti-TH (1:2000) (both from Sigma-Aldrich), goat anti-CRHR1 (1:100), rabbit anti-CRHR2 (1:250), rat anti-dopamine transporter (DAT, 1:200), chicken anti-MAP2 (1:5000) (all from Abcam, Cambridge, Mass.), rabbit anti-Nurr1 (1:100, Santa Cruz Biotechnology), mouse anti-Nurr1 (1:100, R&D Systems, Minneapolis, Minn.), rabbit anti-TH (1:1000), mouse anti-NeuN (1:200) (both from Chemicon, Temecula, Calif.), rabbit anti-acetyl-histone H3 (1:1000, Upstate Biotechnology, Lake Placid, N.Y.), rabbit anti-Foxa2 (1:500, Cell Signaling Technology), and rabbit anti-Pitx3 (1:100, Invitrogen).

For detection of dopaminergic neurons, the bound anti-TH antibody was visualized by incubation with an appropriate biotinylated secondary antibody followed by the Vectastain avidin-biotin-peroxidase (ABC) reagents (Vector Laboratories, Burlingame, Calif.) and color development with 3,3′-diaminobenzidine. The number of TH-positive neurons were counted in the entire surface area of a culture well or coverslip. For fluorescent double-labeling experiments, cells were incubated for 1 h at room temperature with rhodamine-conjugated donkey anti-mouse IgG (1:250), FITC-conjugated goat anti-rabbit IgG (1:250) or FITC-conjugated donkey anti-goat IgG (1:200) secondary antibodies (all from Jackson Immuno Research, West Grove, Pa.). Samples were counter-stained with Hoechst 33342 (5 μg/mL, Sigma-Aldrich) or DAPI (1 μg/mL), and mounted with 50% glycerol in PBS. Microscopic observations were done with a Zeiss Axiovert 200 M fluorescent microscope or a Zeiss LSM 510 META confocal imaging system (Carl Zeiss, Oberkochen, Germany). Immuno-reactive or Hoechst 33342-stained cells were counted in at least 10 random areas of each culture coverslip at a magnification of 100×.

### Immuno-Histochemistry

For immuno-histochemistry, rat embryos were perfused with 4% paraformaldehyde, cryopreserved with 30% sucrose in PBS overnight, embedded in Tissue Tek OCT compound (Sakura Finetek, Torrance, Calif.), and then frozen at −80° C. For Nurr1 staining, the embryos were perfused with 4% paraformaldehyde/0.15% picric acid followed by soaking in the same solution overnight at 4° C. Coronal sections (16 μm thick) were blocked with 2% FBS and 3% normal goat or normal donkey serum for 1 h at room temperature, and subsequently incubated with primary antibodies overnight at room temperature. Primary antibodies were rabbit anti-Lmx1b (1:3000), guinea-pig anti-Lmx1b (1:1500), rabbit anti-UCN (1:200), goat anti-CRHR1 (1:100), rabbit anti-CRHR2 (1:250), mouse anti-Nurr1 (1:100), rabbit anti-Nurr1 (1:100), and mouse anti-TH (1:2000). After washing with PBS, secondary antibodies conjugated to the FITC or rhodamine were applied to sections for 1 h. Sections were examined with fluorescent microscope or confocal microscope. Quantitative cell counts were performed in every third section through the entire A9-A10 populations in 5-10 embryos per condition.

### Immuno-Precipitation

For immuno-precipitation, VM precursor cells were treated with UCN for 24 hours and then harvested with M-PER® Mammalian Protein Extraction Reagent (Pierce, Rockford, Ill.) containing 1 mM PMSF, 10 μg/mL aprotinin, 10 μg/mL leupeptin, and 5 μg/mL pepstatin A. The cytosolic lysates (250 μg of protein) were incubated with a rabbit anti-Nurr1 antibody (Santa Cruz Biotechnology) with gentle rocking overnight at 4° C. PureProteome™ protein G magnetic beads (Merck Millipore, Billerica, Mass.) were added (15 μL of suspension) and rotated for 3 h at 4° C. After washing the beads with ice-cold immuno-precipitation buffer (20 mM Tris, pH 7.5, 150 mM NaCl, 1 mM EDTA, 1 mM EGTA, 1% Triton X-100, 2.5 mM sodium pyrophosphate, 1 mM β-glycerolphosphate, 1 mM Na₃VO₄, 1 μg/mL leupeptin, 1 mM PMSF), immuno-precipitated proteins were eluted in sample buffer, and subjected to Western blot analyses with anti-MeCP2, -CoREST, -HDAC1, -Nurr1, -Foxa2, or -Pitx3 antibodies.

### Western Blot Analysis

For Western blot analysis, VM precursor cells were lysed in M-PER® Mammalian Protein Extraction Reagent (Pierce). Protein concentration was determined by Bradford assay (Bio-Rad, Hercules, Calif.). 30˜50 μg of protein sample was separated on 10˜12% sodium dodecyl sulphate-polyacrylamide gel (SDS-PAGE) and transferred to immobilon polyvinylidene difluoride (PVDF) membranes (Merck Millipore). The membranes were incubated in Tris-buffered saline (TBST, 0.1 M Tris/HCl, pH 7.4, 0.9% NaCl, 0.1% Tween 20) supplemented with 5% non-fat dry milk for 1 h to block nonspecific binding. After rinsing with TBST buffer, the membranes were incubated with the following antibodies: rabbit anti-acetyl histone H3 (1:2500), rabbit anti-Nurr1 (1:250), rabbit anti-CoREST (1:1000, Upstate Biotechnology), mouse anti-HDAC1 (1:1000), rabbit anti-MeCP2 (1:1000), rabbit anti-Foxa2 (1:1000) (all from Cell Signaling Technology), rabbit anti-Pitx3 (1:1000, Abcam), mouse anti-β-actin (1:4000, Sigma-Aldrich). The membranes were washed three times with TBST followed by incubation with appropriate horseradish peroxidase-conjugated secondary antibodies. The antigen-antibody complex was detected by using an ECL chemi-luminescence detection system (PerkinElmer, Boston, Mass.). The intensity of the bands was quantified with a GS-800 calibrated densitometer (Bio-Rad), and calculated as the optical density X area of bands.

### TH Promoter Assay

The luciferase reporter vector pTH6.0-GL3 constructed by engineering the 6.0 kb upstream sequences of the rat TH (TH6.0) gene into pGL3 was kindly provided by Dr. Kwang-Soo Kim (Harvard Medical School, Belmont, Mass.). Vector expressing Nurr1 (mouse) was constructed by inserting the respective cDNA into pAAV-MCS vector (pAAV-Nurr1), in which the cloned gene is designed to be expressed under the control of the cytomegalovirus (CMV) immediate-early promoter. pTH6.0-GL3 alone or combined with pAAV-Nurr1 were transfected into a human neuro-blastoma SH-SY5Y cell line or cortical NPCs, respectively, using Lipofectamine 2000 (Invitrogen) according to the protocol of the manufacturer. For determine the effect of MeCP2-CoREST-HDAC1 repressor complex on TH promoter activity, the SH-SY5Y cells were double transfected with pTH6.0-GL3 and pCMV6-Entry-Mecp2 (Flag-tagged MeCP2)/or pCMV6-Entry-Rcor1 (Flag-tagged CoREST) (both from OriGene Technologies, Rockville, Md.)/or pK7-HDAC1-GFP (Addgene). At 24 h after transfection, cells were treated with 1 μM UCN or 1.25 mM SB for another 24 h. Luciferase activity of cell lysates was determined luminometrically by the luciferase assay system (Promega, Madison, Wis.) as specified by the manufacturer. Luciferase activity was normalized to the protein content of the extracts. Relative luciferase activity was determined to reflect promoter activity of TH, expressed as the fold increase relative to the activity of control or untreated mock control.

### DA Uptake Assay

For DA uptake assay, cells were washed twice with warm Krebs-Ringer buffer (16 mM sodium phosphate, 119 mM NaCl, 4.7 mM KCl, 1.8 mM CaCl₂, 1.2 mM MgSO₄, 1.3 mM EDTA and 5.6 mM glucose, pH 7.4), and then incubated with 1 μM [³H]-dopamine(DA)(60 Ci/mmol, PerkinElmer) in Krebs-Ringer buffer at 37° C. for 20 min. After washing with ice-cold Krebs-Ringer buffer, cells were collected in 1 N NaOH and radioactivity was counted with a liquid scintillation counter. Non-specific uptake was determined in parallel wells that received both the tritiated tracer and 10 μM mazindol. The specific [³H] DA were calculated by subtracting the amount of radioactivity obtained in the presence of mazindol from that obtained in the absence of mazindol.

### UCN Assay

For UCN assay, protein was extracted from VM tissues with an M-PER® Mammalian Protein Extraction Reagent (Pierce) containing protease inhibitors. Protein extracts were kept frozen in aliquots at −80° C. until use. UCN content was measured with a commercial enzyme immunoassay assay (EIA) kit (Phoenix Pharmaceuticals, Burlingame, Calif.) according to the manufacturer's instructions.

siRNA Transfection

Rat Accell™ SMARTpool Foxa2 and Pitx3 siRNA were obtained from Dhmarcon (Thermo Scientific, Lafayette, Colo.). Nonspecific siRNA was used as negative control. The target sequences of the rat-specific siRNA used herein were as follows.

For Foxa2 siRNA
(SEQ ID NO: 45)
CUGUCAUUCUAAAUAGGGA,
(SEQ ID NO: 46)
GGGUUGUAUUGAUGUUUAA,
(SEQ ID NO: 47)
GGGUCUGAUUUAAUUUAUG
and
(SEQ ID NO: 48)
CCAUGUAGUUUUAACAGAA;
For Pitx3 siRNA:
(SEQ ID NO: 49)
CCUUCAACUCGGUCAACGU,
(SEQ ID NO: 50)
CUCCUCCCCUUAUGUAUAC,
(SEQ ID NO: 51)
GCGCUGUCAUUGUCAGAUG
and
(SEQ ID NO: 52)
UUCACAACCUGUAUUCUCA.

VM precursor cells were seeded in 24-well plates for 4 hours prior to transfection. siRNA duplexes were transfected into VM precursor cells using Accell™ siRNA delivery system according to the manufacture's instruction. After 24 hours of transfection, VM precursor cells were transfected with pTH6.0-GL3 followed by treatment with UCN, and luciferase activity was assayed. For TH⁺ cell counting analysis or verification of Foxa2 and Pitx3 knockdown efficiency, Cells were exposed to 1 μM UCN for 2-3 days after siRNA transfection.

### Expression of UCN and CRHRs in the Developing VM and in VM Precursor Cultures

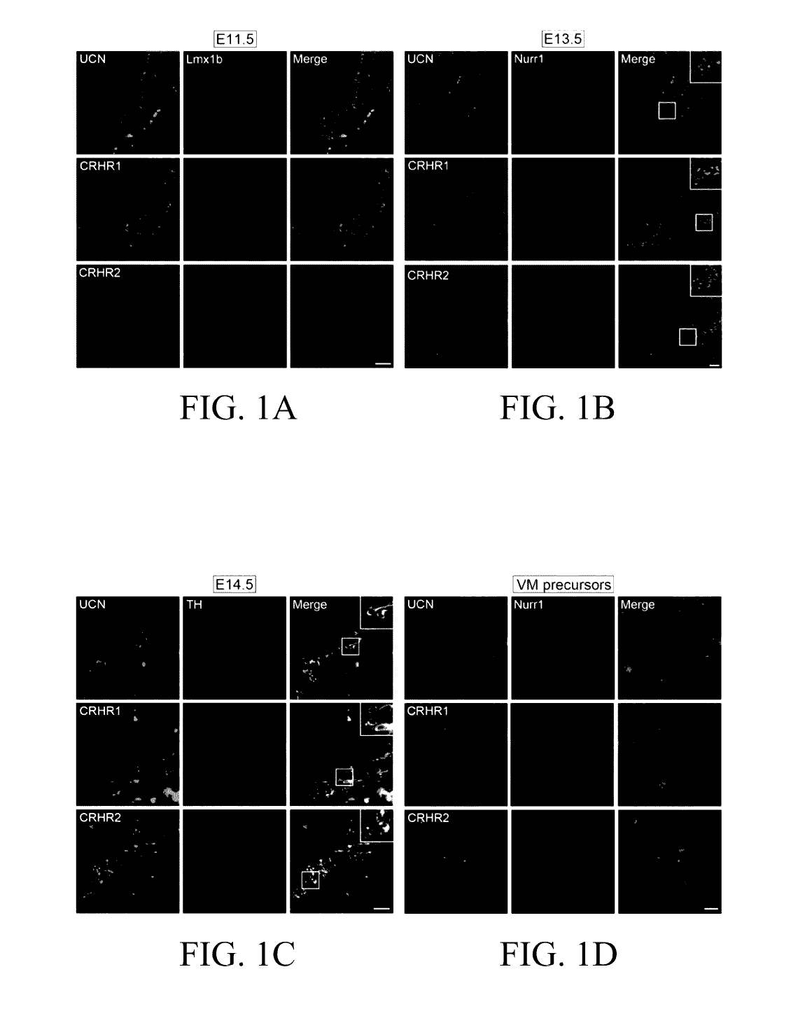

To establish whether UCN plays a physiological role in the development of mDA neurons in vivo, DA precursor cells were first examined for the expression of UCN and its receptors. Referring to FIG. 1A, UCN and its receptors CRHR1 and CRHR2 were found to be expressed in all midbrain alar and basal plate progenitors at this stage in rat E11.5 brains. However, low levels of UCN and CRHRs were detected in the floor plate, in which the mDA progenitors are characterized by Lmx1b immuno-staining.

Referring to FIG. 1B, at a later embryonic stage (E13.5), UCN and CRHRs were expressed in broader regions, whereas a weak expression was observed in the ventricular zone of the VM. Double immuno-histochemistry shows that UCN and CRHRs were largely co-localized with Nurr1, a post-mitotic DA progenitor marker.

Referring to FIG. 1C, UCN and CRHRs were also expressed in TH⁺ mature DA neurons (TH: tyrosine hydroxylase, a DAergic marker) at E14.5. Referring to FIG. 1D, immuno-staining experiments reveal that UCN and CRHRs were expressed in VM precursor cultures and was co-expressed with Nurr1. The prominent expression of UCN and CRHRs in the developing VM, and in cultured VM precursor cells, implies their putative involvement in the development of mDA neurons.

### UCN Promotes DA Neuron Differentiation of VM Precursor Cultures

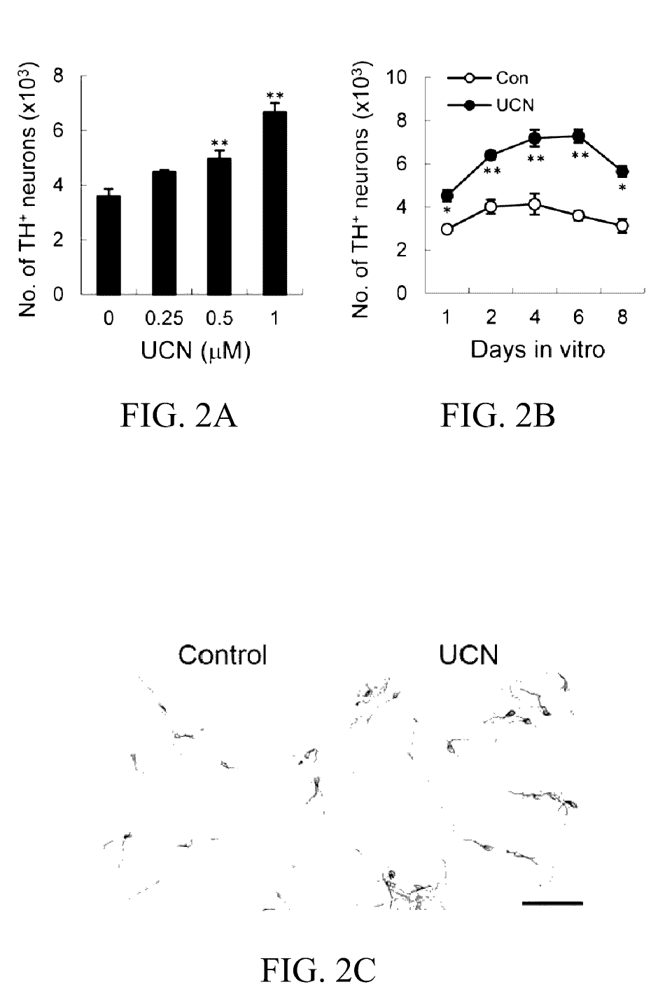

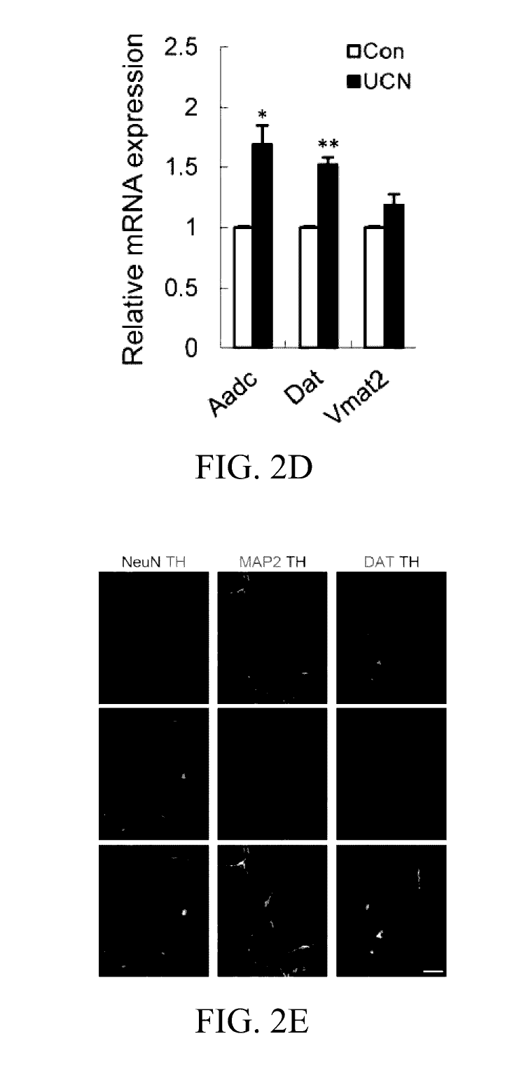

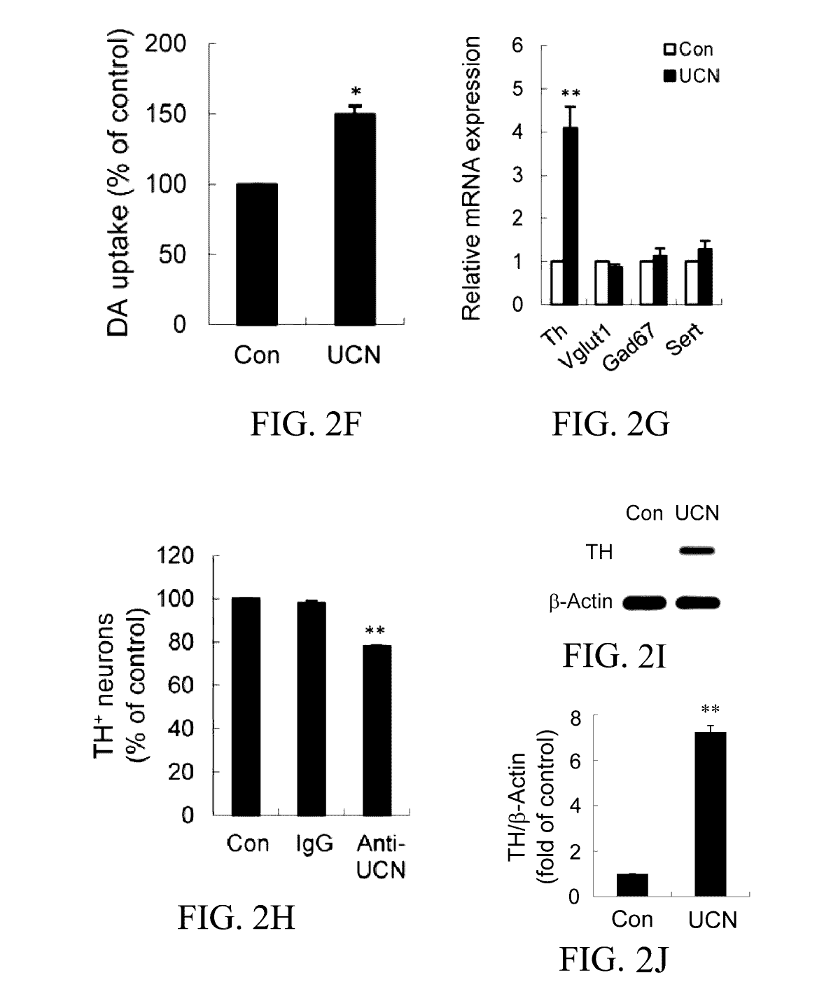

In order to determine whether UCN treatment promotes DA neuron differentiation, rat VM precursor cultures were treated with increasing doses of UCN or control medium for 3 days. FIG. 2A shows treatment with UCN resulted in a dose-dependent increase in numbers of TH⁺ cells. Furthermore, FIGS. 2B and 2C show the effect of 1 μM UCN as early as 1 day in vitro was clearly higher than that of control medium at 4 days in vitro, and start to diminish thereafter in control cultures.

Additionally, FIG. 2D shows the result of the expression of DA phenotype genes in VM precursor cultures by real-time PCR. In UCN-treated VM precursor cultures, trends were found for increased dopamine transporter (DAT) and L-aromatic amino acid decarboxylase (AADC) mRNA, confirming the increase in DA neurons.

As shown in FIG. 2E, all the TH⁺ cells in the UCN-treated cultures also expressed proteins specific for neurons (NeuN and MAP2) and DA homeostasis (DAT). As shown in FIG. 2F, consistent to the increase of DAT mRNA expression by UCN treatment, DAT-mediated specific DA uptake was significantly enhanced in UCN-treated cultures. These findings suggest that UCN does not merely activate TH gene expression but promotes DA neuron differentiation of VM precursor cultures.

Referring to FIGS. 2G and 2H, UCN action was specific for DA neuron differentiation.

Differentiation phenotypes of VM precursor cultures were determined by real-time RT-PCR analyses for DAergic (TH), glutamatergic (VGULT1), GABAergic (GAD67), and serotonergic (SERT) neuron-specific markers. As shown in FIG. 2G, UCN significantly elevated expression of TH mRNA but has no effect on mRNA expression specific for glutamatergic, GABAergic and serotonergic neurons. Having established that UCN was present in mesencephalic cells, FIG. 2H further elucidates whether endogenous UCN contributes to the acquisition of the TH-positive phenotype. Addition of neutralizing anti-UCN antibody significantly reduces the number of TH⁺ neurons in precursor cultures, indicating that endogenous UCN was involved in the induction of the TH-positive phenotype. Furthermore, FIG. 2I shows that UCN also induced the increases of TH protein levels in human DA neuro-blastoma SH-SY5Y cells.

UCN Facilitates the Maturation of Nurr1—Expressing Precursors into DA Neurons

Nurr1 (NR4A2) is the DA key fate-determining transcription factor which expressed in late mDA neuronal progenitors. The instructive mechanisms underlying the UCN effect could be speculated to initiate DA specification of VM-NPCs with induction of Nurr1 expression or to promote the differentiation of Nurr1⁺ progenitor cells into the mature DA phenotype.

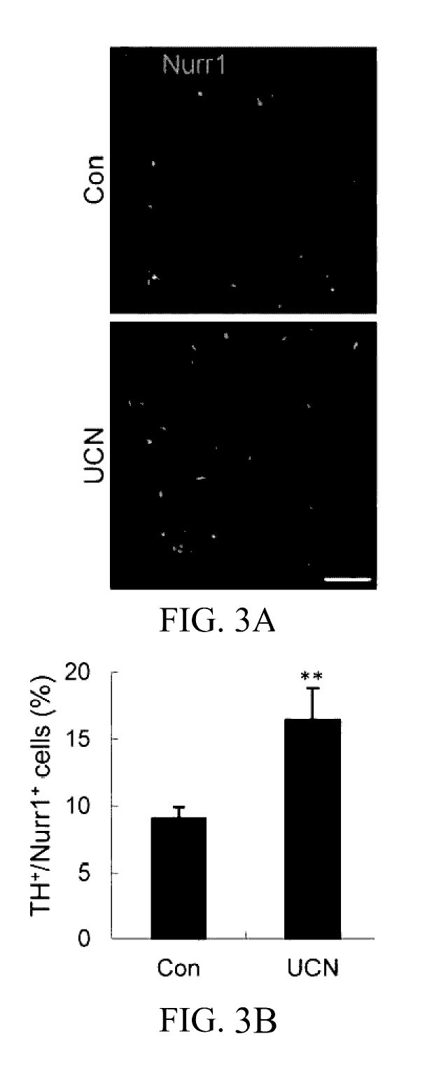

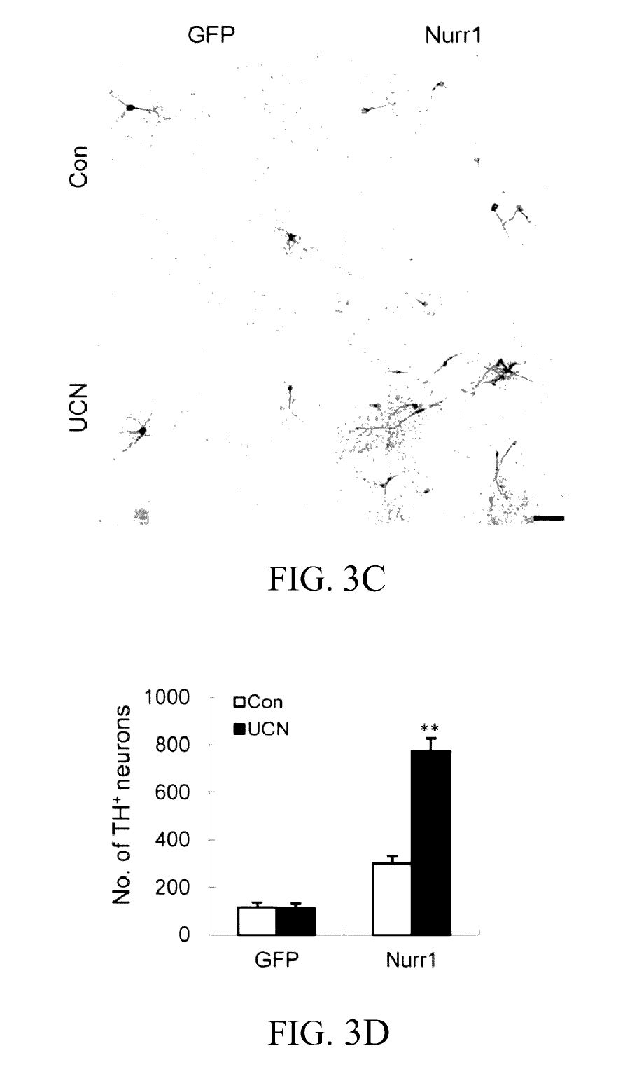

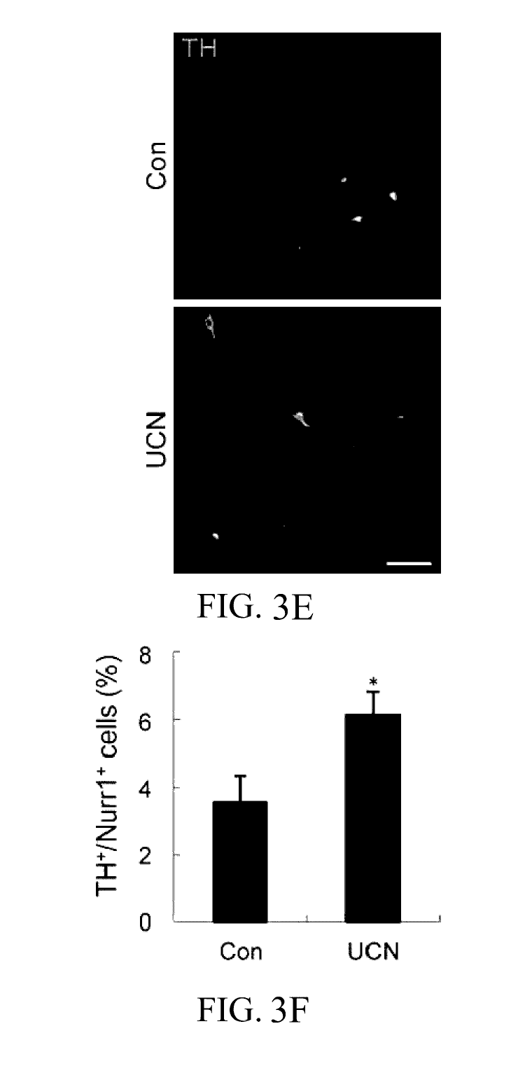

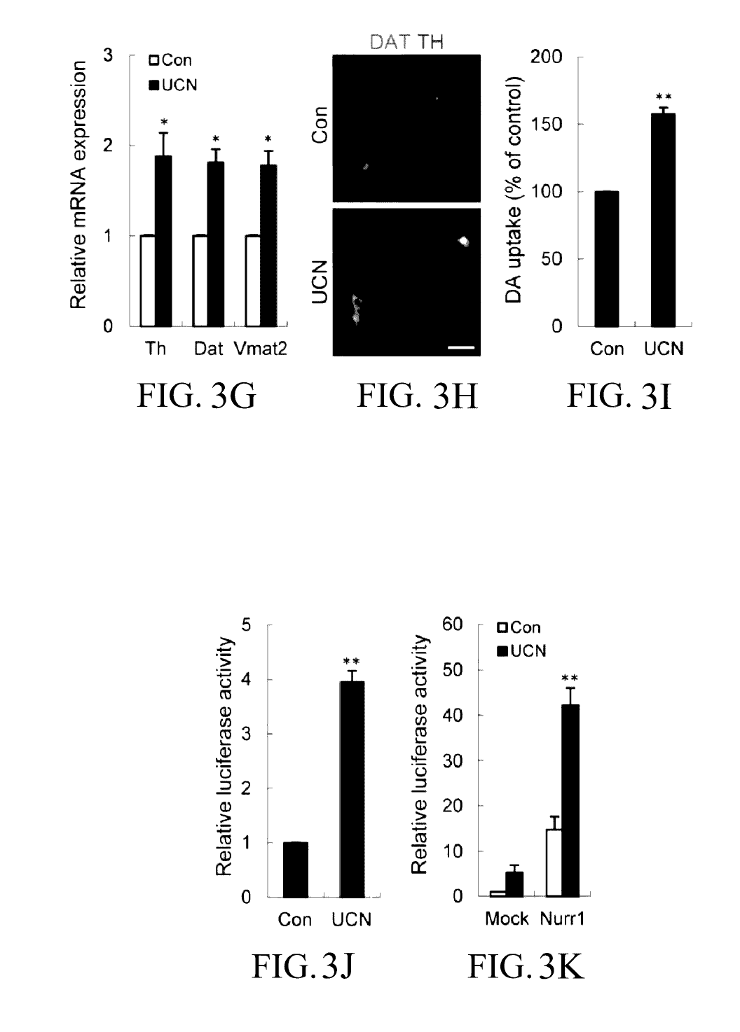

First, the inventors found that the number of Nurr1⁺ cells was not changed upon treatment with UCN (4.91%±0.54% in control vs. 4.68%±0.4% in UCN treated cultures). Next, the differentiation of Nurr1⁺/TH⁻ precursors into Nurr1⁺/TH⁺ DA neurons was examined. FIG. 3A shows that in UCN treated cultures, more Nurr1⁺ cells develop concomitant TH⁺ expression, compared to controls. To further confirm the increase of Nurr1 precursor maturation by UCN, the Nurr1 gain-of-function assays were performed. Referring to FIG. 3B, only a few TH⁺ cells were differentiation from non-dopaminergic cortical neural precursor cells (NPCs) transduced with GFP and UCN treatment do not change TH⁺ cell numbers in these cultures. The increase in TH⁺ cell numbers following UCN exposure was observed in the cortical NPCs transduced with Nurr1. In addition, FIG. 3C shows UCN treatment increased the conversion of Nurr1⁺ cortical NPCs into DA neurons. As shown in FIG. 3D, exposure of exogenous Nurr1-expressing NPCs to UCN increased gene expression associated with the DA phenotype (TH, DAT and VMAT2). Also, as shown in FIG. 3E, almost all of TH⁺ cells expressed the mature DA neuron-specific marker DAT. Additionally, as shown in FIG. 3F, UCN-enhanced cells differentiated with Nurr1-transduced cultures displayed increased DA neuronal function as assessed by DA uptake.

To determine whether UCN-induced increase in TH is mediated by upregulating TH trans-activation, a rat TH promoter/reporter construct pTH6.0-GL3 is used to analyze TH promoter activity. Referring to FIGS. 3G and 3H, consistent with the increase in TH mRNA levels by UCN, UCN treatment of SHSY5Y cells or Nurr1-transfected cortical NPCs resulted in activation of the TH promoter. Collectively, these findings suggest that UCN increases the number of DA neurons by a mechanism that involves promoting the maturation of DA precursors and the acquisition of a neuronal DA phenotype.

### Histone Deacetylase Inhibition Mediated UCN-Enhanced DA Neuron Differentiation

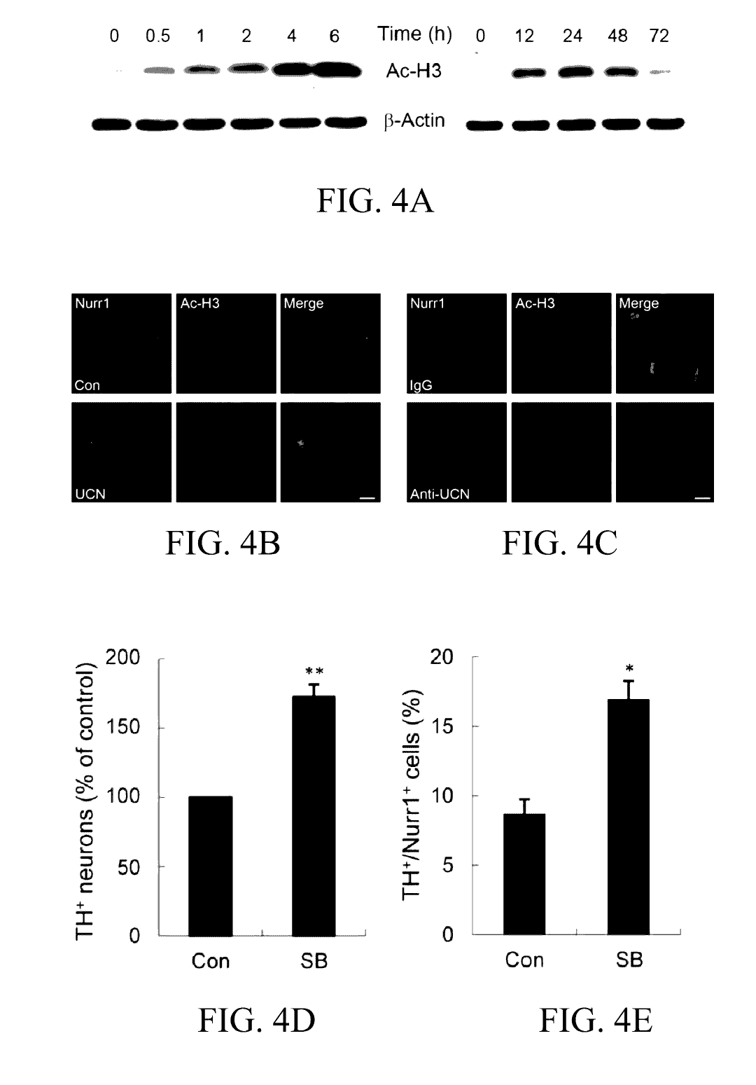

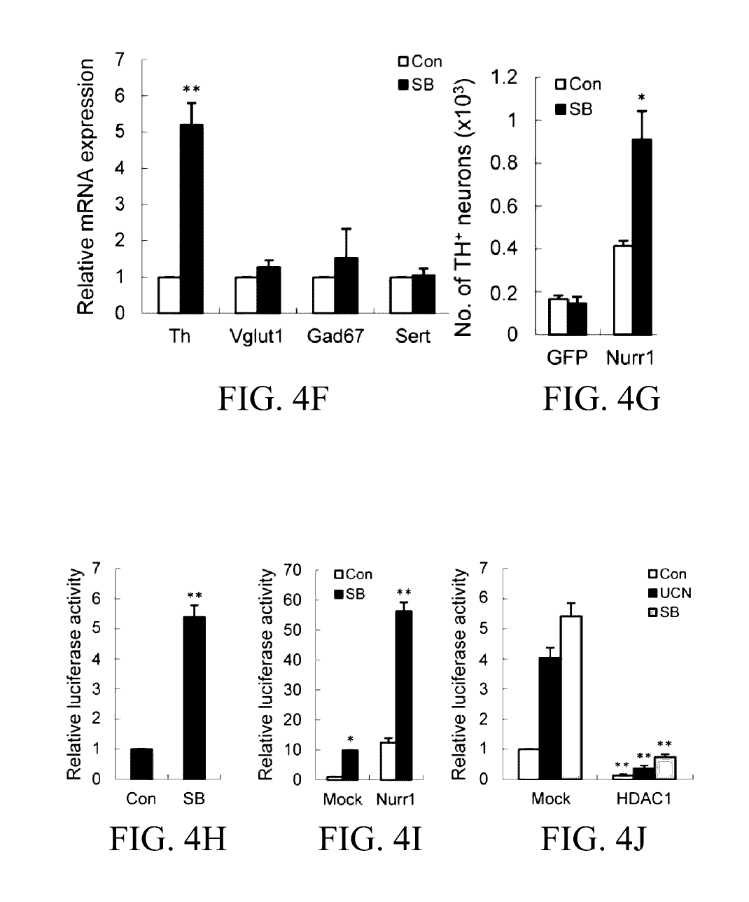

The deacetylated state of histones near the transcription start site (TSS) is responsible for the repression of gene transcription. The potential involvement of histone deacetylase (HDAC) in the regulation of Nurr1 transcriptional activity was reported previously (Jacobs et al., 2009). To determine whether changes in histone acetylation occur during UCN treatment, protein extracts were harvested from VM precursors and analyzed by Western blot analysis with antibody specific for acetylated histone H3 (Ac-H3) in the present invention. Referring to FIG. 4A, UCN significantly increases levels of Ac-H3 within minutes and histone H3 acetylation is lasted for at least 48 hours with a single application of UCN.

Similarly, referring to FIG. 4B, UCN-mediated acetylation of histone H3 was also observed at the cellular level that Ac-H3-immuno-reactivity was increased in either non- or Nurr1-expressing cells. Furthermore, referring to FIG. 4C, incubation with anti-UCN antibody decreases intracellular Ac-H3 levels. These findings demonstrate that either exogenous or secreted UCN could induce histone H3 acetylation in Nurr1⁺ DA precursors.

To examine whether HDAC inhibition is a critical mechanism in the UCN induced DA neuron differentiation, a HDACs inhibitor SB was used to mimic the effects of UCN. As shown in FIG. 4D, treatment with SB resulted in an increase in cells with TH staining compared with control cultures. As shown in FIG. 4E, inhibition of HDACs by SB increased DA cell numbers was through conversion of Nurr1⁺ precursors into DA neurons. Referring to FIG. 4F, similar to the UCN action, TH mRNA expression, but not VGLUT1, GAD67, and SERT mRNA, was markedly upregulated in SB-treated cultures. Furthermore, FIG. 4G shows that the increase in TH⁺ cell numbers following SB exposure was also observed in Nurr1-transduced cortical NPCs. FIGS. 4H and 4I show that SB treatment was also shown to increase TH promoter activity in SH-SY5Y cells and Nurr1-transfected cortical NPCs. HDAC1 has been shown to be a critical repressor protein in induction of TH. To further confirm that the effects of UCN were mediated through HDAC inhibition, the effect of overexpression of HDAC1 on blocking of UCN-induced TH expression was examined. FIG. 4J shows UCN- or SB-elicited TH promoter activity was significantly attenuated by co-transfection with HDAC1. These data taken together suggest that UCN-induced increase in DA neuron differentiation is dependent on HDAC inhibition.

### Epigenetic Events at the TH Promoter Caused by UCN

In terminally differentiated mDA neurons, TH is generally used to mark DA neurons and is present in mature DA cells. As mentioned above, UCN is shown to induce TH mRNA expression and TH promoter activity. To provide further support that UCN modulates Nurr1-mediated endogenous TH transcription, a chromatin immuno-precipitation (ChIP) assay was performed to confirm UCN action on the interactions of Nurr1 with the rat TH promoter region.

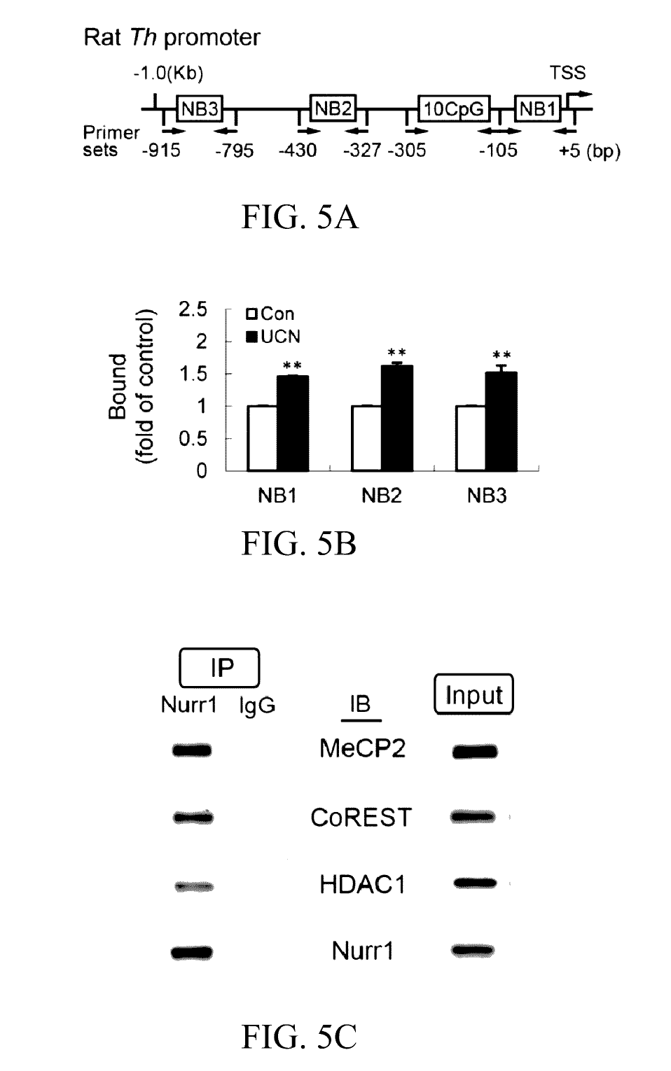

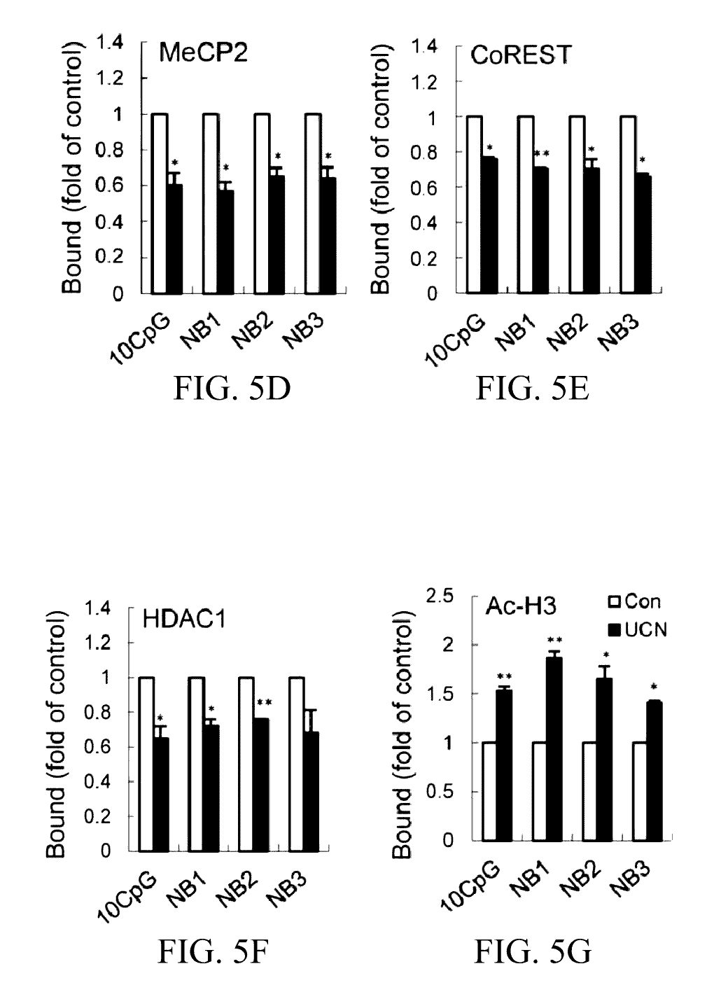

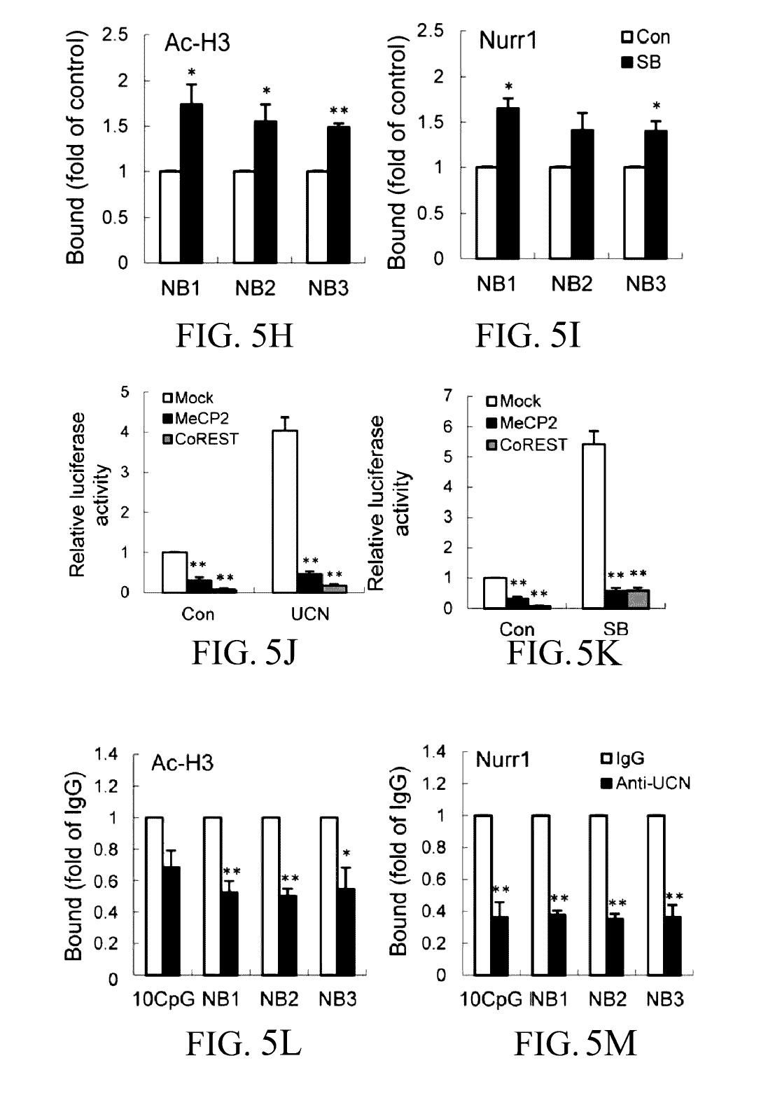

FIG. 5A depicts schema of rat TH promoter. There are three regions predicted to be Nurr1 binding sits (NB1-3) within −1 kb of the transcription start site (TSS) of the rat TH promoter. FIG. 5B shows ChIP analysis to determine Nurr1 protein enrichment at the TH promoter. ChIP assays in VM precursor cultures demonstrated the Nurr1 protein occupancy in those promoter regions was significantly increased by UCN treatment. This illustrates that UCN may exert an epigenetic modulation function to promote TH gene transcription by enhancing Nurr1 access to the promoter regions. The activity of nuclear receptors is highly regulated by co-regulating proteins. Upon activation of nuclear receptors, co-repressors are released and co-activators are recruited. Possibly, UCN treatment affects the composition of the Nurr1 transcriptional complex. FIG. 5C is the result of immuno-precipitation (IP) assays showing that methyl CpG binding protein 2 (MeCP2, an epigenetic repressor), element-1 silencing transcription factor corepressor (CoREST), and HDAC1 (a common component of the CoREST- and MeCP2-mediated epigenetic repressor complex) associated with Nurr1. It indicated that these proteins form a complex in VM precursors and may affect the Nurr1 transcriptional activity. FIGS. 5D to 5G are the results of ChIP assays for MeCP2, CoREST, HDAC1 and Ac-H3, respectively. It reveals that UCN treatment significantly reduced MeCP2 recruitment to the TH promoter region of Nurr1-binding sites and repeated CpGs. Interestingly, it is observed that MeCP2 was also recruited to NB2-containing region. MeCP2 may be indirectly recruited to the NB2-containing region by intermediary protein-protein associations, such as via Nurr1.

Similar to MeCP2, as shown in FIG. 5E, decreased CoREST binding following UCN exposure is also evident in the TH promoter regions. Additionally, HDAC1 is a common component of the CoREST- and MeCP2-mediated epigenetic repressor complex. Referring to FIG. 5F, HDAC1 recruitment was significantly reduced in the presence of UCN. Consequently, referring to FIG. 5G, histone H3 acetylation, a histone modification associated with open chromatin structures, was significantly increased at TH promoter regions in UCN-treated cultures.

Furthermore, FIG. 5H shows that UCN-mediated regulation of Nurr1 recruitment and histone acetylation could be mimicked by SB. To further confirm that the releasing of the epigenetic repressor complex from the TH promoter contributes to the action of UCN, the effect of overexpression of MeCP2 or CoREST on blocking of UCN-induced TH expression was examined.

FIGS. 5I and 5J show the results of overexpression of MeCP2 or CoREST on blocking of UCN-induced TH expression. It further confirms that the releasing of the epigenetic repressor complex from the TH promoter contributes to the action of UCN. The results show that forced MeCP2 or CoREST expression resulted in attenuation of UCN or SB-elicited TH promoter activity.

As shown in FIG. 5K, neutralization of secreted UCN indeed reduced the basal occupancy of Ac-H3 and Nurr1 at the TH promoter. These findings collectively suggest that UCN keep promoters in an acetylated state by decreasing the association of MeCP2-CoREST-HDAC1 repressor complex with the promoters to activate the transcription of TH gene.

### Upregulated Foxa2 and Pitx3 are Involved in UCN MediatedDA Neuron Differentiation

Several characterized transcription factors, including Nurr1, Pitx3, En, Lmx1a/b, and Foxa2, are involved in the maturation of post-mitotic DA neurons. The expressions of these intrinsic determinants following UCN exposure were analyzed in the present invention.

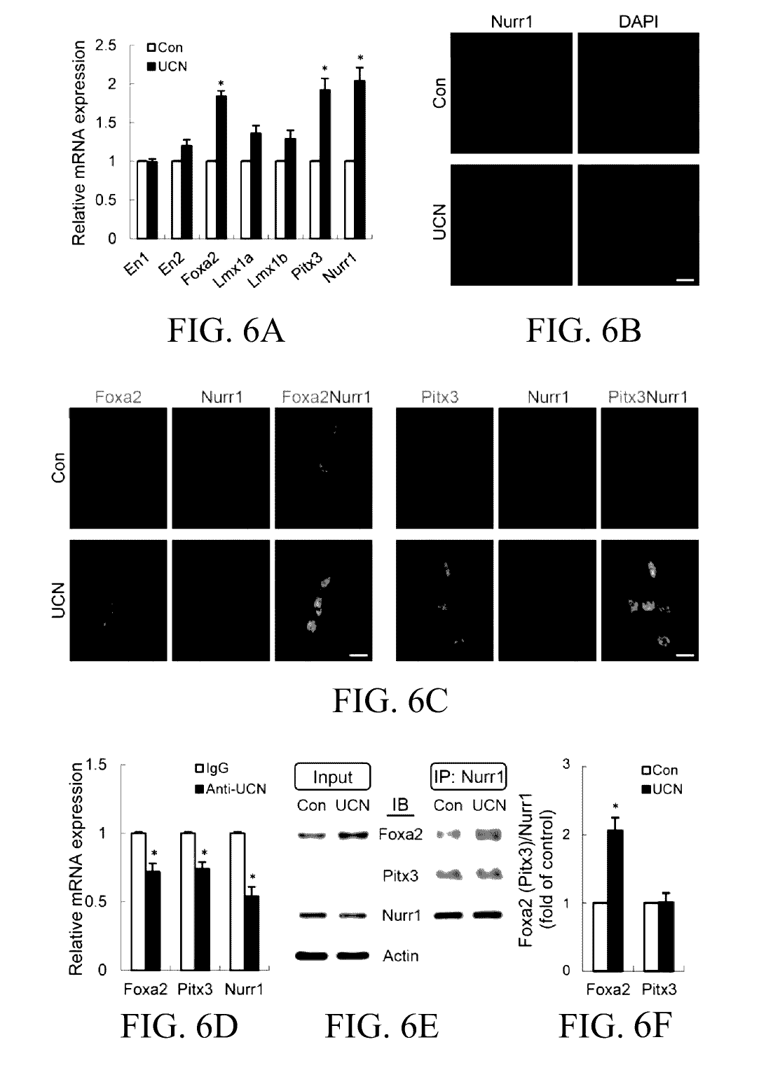

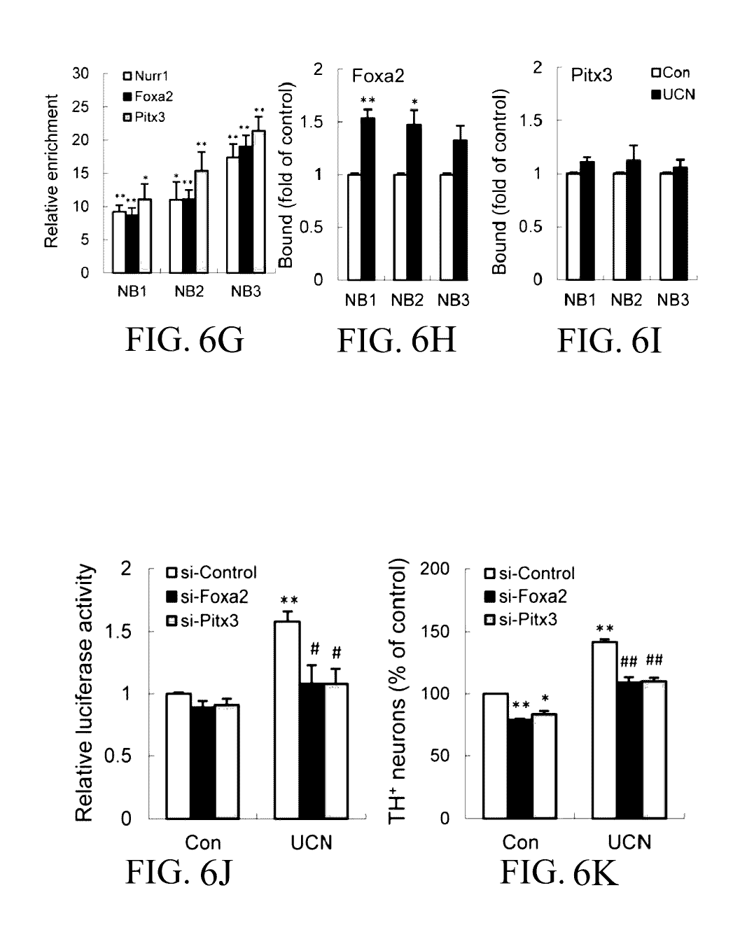

FIG. 6A shows the result of real-time RT-PCR analysis. It reveals that Nurr1, Foxa2 and Pitx3 were upregulated by UCN. Referring to FIGS. 6B and 6C, the Nurr1-immuno-reactive intensity is increased following UCN exposure and most of the Nurr1+ cells in the UCN-treated cultures appear to express an increased level of Foxa2 and Pitx3. Referring to FIG. 6D, blocking of endogenous UCN by anti-UCN antibody was shown to down-regulate the basal expression of Nurr1, Foxa2, and Pitx3 genes. These data demonstrate that endogenous UCN acts as an inducer to upregulate late transcription factors expression during late DA neuron differentiation in the VM.

It is known that both Pitx3 and Foxa2 proteins interact physically with Nurr1 and generate a functional protein complex on DA phenotype gene promoters. The effect of UCN on the physical protein-protein interaction between Nurr1 and these two proteins was examined in the present invention. FIG. 6E shows that in VM precursor cultures, Foxa2 and Pitx3 proteins are co-precipitated with Nurr1 in IP assays. The amount of Foxa2 that precipitates with Nurr1 was markedly enhanced by UCN, whereas the interaction between Pitx3 and Nurr1 proteins is not significantly altered. As shown in FIGS. 6F and 6G, ChIP analyses show both Foxa2 and Pitx3 proteins were enriched in the Nurr1 binding sites of TH promoter, and UCN treatment increases Foxa2 but not Pitx3 occupancy. These findings suggest that upregulated Foxa2 and Pitx3 may contribute to UCN-induced DA neuron differentiation.

To investigate the role of Foxa2 and Pitx3 in UCN-induced DA neuron differentiation, in the present invention the expression of Foxa2 and Pitx3 were knocked down by using specific small interfering RNA. FIGS. 6H and 61, show that down-regulation of Foxa2 or Pitx3 in VM precursor cultures decreased UCN-induced TH promoter activity, and reduced DA neuronal yields.

### UCN Increases DA Neuron Differentiation in Embryonic VM

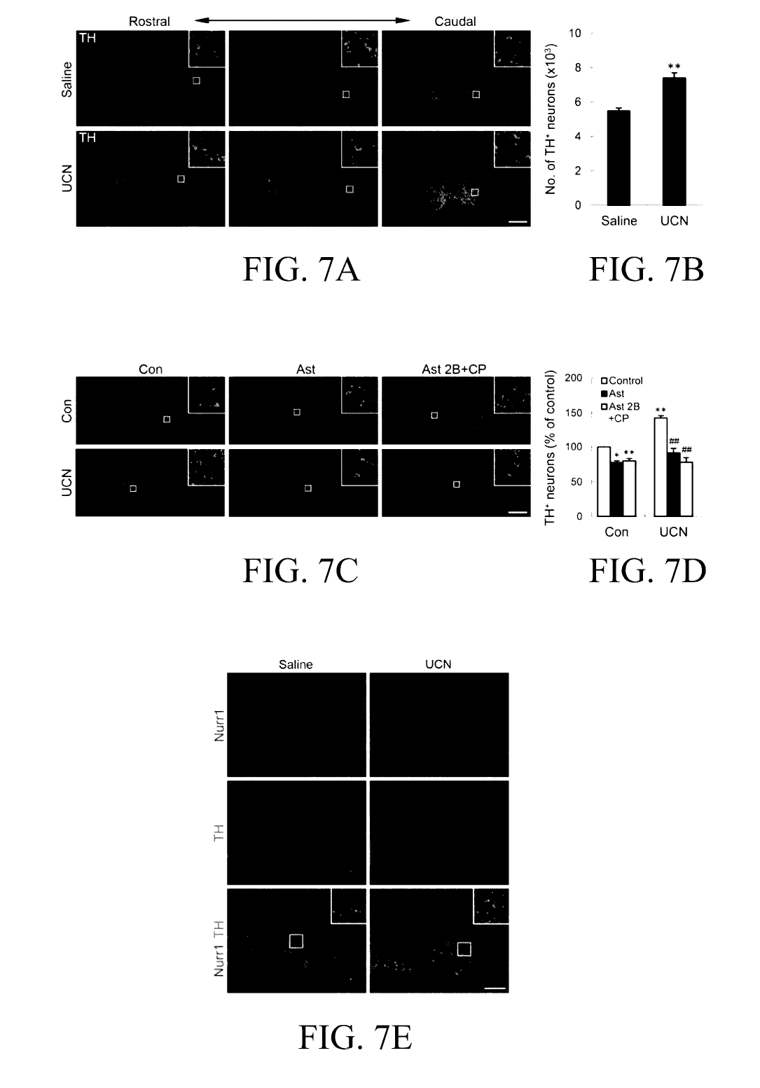

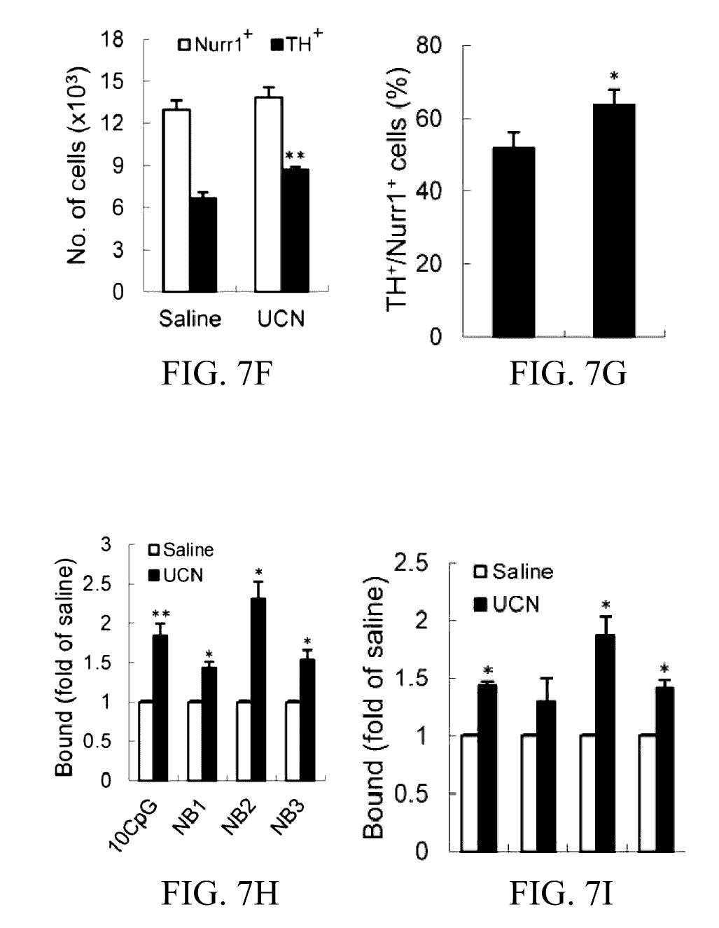

To further understand the physiological relevance of the UCN-mediated differentiation of DA neurons in vivo, the E16.5 rat embryo is examined to observe the effect of UCN administration on differentiation in the VM in the present invention. FIG. 7A illustrates midbrain TH-immuno-reactive neurons in embryos. Further, as shown in FIG. 7B, increased number of ventral mesencephalic TH+ cells is observed in the UCN-treated embryos.

Cultured embryonic midbrain explants, where neurogenesis occurs as in normal midbrain development, have been widely used to study differentiation and neurite growth of DA neurons in the art, for example as described by Baizabal and Covarrubias (2009) and Lin et al. (2005).

Therefore, in the present invention, isolated embryonic midbrain explants ex vivo are treated with CRHRs antagonists in the absence or presence of exogenous UCN. As shown in FIGS. 7C and 7D, either astressin (a peptide CRHRs-nonselective antagonist) or CP-376395 (a non-peptide CRHR1-selective antagonist) in combination with astressin 2B (a peptide CRHR2-selective antagonist) can reduce the TH+ cell numbers in the VM of midbrain explants without exogenous UCN. UCN has been demonstrated to be temporally expressed in the developing VM. These data indicate that the endogenous UCN/CRHRs system plays an active role in this process.

Furthermore, referring to FIGS. 7C and 7D again, UCN treatment increases the number of TH⁺ cells and CRHRs antagonists completely abolish UCN-enhanced DA neuronal yields, which demonstrate the direct effects of UCN on DA neuron differentiation in the developing VM.

Next, the present invention examines the mechanism by which UCN increased the number of DA neurons in vivo in embryos received UCN. As shown in FIGS. 7E and 7F, there is no significant difference in the Nurr1+ cell numbers between saline and UCN-treated embryos. However, it is observed in FIGS. 7E and 7G that a significant proportion of the Nurr1+ cells differentiate into TH+ neurons in UCN treated embryos compared with saline-treated embryos, which suggests that UCN enhances the number of TH+ cells by increasing the differentiation of Nurr1+ precursors into TH+DA neurons.

To further confirm whether UCN-induced epigenetic modulation is occurred in vivo, the present invention examined Nurr1 protein occupancy and histone modification at TH promoter by ChIP analyses using chromatin isolated from the VM of E15.5 embryos following UCN administration. FIG. 7H shows a graph of ChIP assays which revealed that histone H3 acetylation is significantly increased in TH promoter regions by UCN. In addition, referring to FIG. 7I, the same UCN application induces more abundant Nurr1 binding to the TH promoter regions encompassing NB2, NB3 and repeated CpGs. These findings indicate that in addition to direct protein-DNA binding to consensus sequences, Nurr1 proteins can be recruited to TH promoter regions indirectly via other proteins. These results support that UCN is a positive regulator of DA neuron differentiation in vivo as well as in vitro.

### Statistical Analysis

All data are expressed as mean±SEM. Data were analyzed by one-way ANOVA followed by Scheffe's test. For paired analyses, t test was used. A p value less than 0.05 was considered statistically significant.

The invention has been described using exemplary preferred embodiments. However, it is to be understood that the scope of the invention is not limited to the disclosed embodiments. On the contrary, it is intended to cover various modifications and similar rearrangement. The scope of the claims therefore should be accorded the broadest interpretation so as to encompass all such modifications and similar arrangements.

The references listed below cited in the application are each incorporated by reference as if they were incorporated individually.

### REFERENCES OF THE INVENTION

Abeliovich A, Hammond R. Midbrain dopamine neuron differentiation: factors and fates. Dev Biol 2007; 304:447-454.
Ang S-L. Transcriptional control of midbrain dopaminergic neuron development. Development 2006; 133:3499-3506.
Baizabal J M, Covarrubias L. The embryonic midbrain directs neuronal specification of embryonic stem cells at early stages of differentiation. Dev Biol 2009; 325:49-59.
Bayatti N, Zschocke J, Behl C. Brain region-specific neuroprotective action and signaling of corticotropin-releasing hormone in primary neurons. Endocrinology 2003; 144:4051-4060.
Castelo-Branco G, wagner J, rodriguez F J et al. Differential regulation of midbrain dopaminergic neuron development by Wnt-1, Wnt-3a, and Wnt-5a. Proc Natl Acad Sci USA 2003; 100:12747-12752.
Farkas L M, Dünker N, Roussa E et al. Transformating growth factor-beta(s) are essential for the development of midbrain dopaminergic neurons in vitro and in vivo. J Neurosci 2003; 23:5178-5186.
Huang H-Y, Lin S-Z, Chen W-F et al. Urocortin modulates dopaminergic neuronal survival via inhibition of glycogen synthase kinase-3β and histone deacetylase. Neurobiol Aging 2011; 32:1662-1677.
Huang H-Y, Liu D D, Chang H-F et al. Histone deacetylase inhibition mediates urocortin-induced antiproliferation and neuronal differentiation in neural stem cells. Stem Cells 2012; 30:2760-2773.
Jacobs F M, van Erp S, van der Linden A J et al. Pitx3 potentiates Nurr1 in dopamine neuron terminal differentiation through release of SMRT-mediated repression. Development 2009; 136:531-540.
Lin L, Rao Y, Isacson O. Netrin-1 and slit-2 regulate and direct neurite growth of ventral midbrain dopaminergic neurons. Mol Cell Neurosci 2005; 28:547-555.
Liu C-D, Chen Y-L, Min Y-L et al. The nuclear chaperone nucleophosmin escorts an Epstein-Ban Virus nuclear antigen to establish transcriptional cascades for latent infection in human B cells. PLoS Pathog 2012; 8:e1003084
Lovejoy D A, Balment R J. Evolution and physiology of the corticotropin-releasing factor (CRF) family of neuropeptides in vertebrates. Gen Comp Endocrinol 1999; 115:1-22.
Oki Y, Sasano H. Localization and physiological roles of urocortin. Peptides 2004; 25:1745-1749.
Pan W, Kastin A J. Urocortin and the brain. Prog Neurobiol 2008; 84:148-156.
Pedersen W A, Wan R, Zhang P et al. Urocortin, but not urocortin II, protects cultured hippocampal neurons from oxidative and excitotoxic cell death via corticotropin-releasing hormone receptor type 1. J Neurosci 2002; 22: 404-412.
Pfisterer U, Kirkeby A, Torper O et al. Direct conversion of human fibroblasts to dopaminergic neurons. Proc Natl Acad Sci USA 2011; 108:10343-10348.
Prakash N, Brodski C, Naserke T et al. A Wnt1-regulated genetic network controls the identity and fate of midbrain-dopaminergic progenitors in vivo. Development 2006; 133:89-98.
Prakash N, Wurst W. Development of dopaminergic neurons in the mammalian brain. Cell Mol Life Sci 2006; 63:187-206.
Ye W, Shimamura K, Rubenstein J L et al. FGF and Shh signals control dopaminergic and serotonergic cell fate in the anterior neural plate. Cell 1998; 93:755-766.

1. A method for enhancing differentiation of dopaminergic (DA) neurons, comprising culturing precursor cells in a DMEM/F12/N2 medium; administering urocortin (UCN) to the precursor cells; and differentiating the precursor cells into the DA neurons.

2. The method according to claim 1, wherein the DA neurons are midbrain DA neurons.

3. The method according to claim 1, wherein the precursor cells are midbrain DA precursor cells, Nurr1-expressing neural precursor cells (NPCs), or DA neuro-blastoma.

4. The method according to claim 1, wherein the differentiation of DA neurons is late differentiation of DA precursor cells.

5. The method according to claim 1, wherein the UCN is administered at an amount of 0.25 μM to 1 μM.

6. The method according to claim 5, wherein the UCN is administered at an amount of 0.5 μM to 1 μM.

7. The method according to claim 6, wherein the UCN is administered at an amount of 1 μM.

8. The method according to claim 1, wherein the precursor cells are administered with the UCN for 1 to 8 days.

9. The method of claim 1, further comprising measuring a level of at least one DA phenotype gene in the precursor cells.

10. The method of claim 9, wherein the at least one of the DA phenotype gene is selected from the group consisting of tyrosine hydroxylase (TH), dopamine transporter (DAT), 1-aromatic amino acid decarboxylase (AADC) and vesicular monoamine transporter (VMAT2).

11. The method of claim 1, further comprising determining an activity of histone deacetylase (HDAC) and measuring a level of acetylated histone H3 (Ac-H3) in the precursor cells.

12. The method of claim 1, further comprising measuring a level of transcription factors Nurr1, Foxa2, and Pitx3 in the precursor cells.

13. A method for producing midbrain dopaminergic (DA) neurons, comprising culturing midbrain DA precursor cells in a DMEM/F12/N2 medium; administering UCN to the midbrain DA precursor cells and differentiating the midbrain DA precursor cells into the midbrain DA neurons.

14. The method according to claim 13, wherein the UCN is administered at an amount of 0.25 μM to 1 μM.

15. The method according to claim 14, wherein the UCN is administered at an amount of 0.5 μM to 1 μM.

16. The method according to claim 15, wherein the UCN is administered at an amount of 1 μM.

17. The method according to claim 13, wherein the midbrain DA precursor cells are administered with the UCN for 1 to 8 days.

18. The method of claim 13, further comprising measuring a level of at least one DA phenotype gene in the midbrain DA precursor cells, wherein the at least one of the DA phenotype gene is selected from the group consisting of tyrosine hydroxylase (TH), dopamine transporter (DAT), 1-aromatic amino acid decarboxylase (AADC) and vesicular monoamine transporter (VMAT2).

19. The method of claim 13, further comprising determining an activity of histone deacetylase (HDAC) and measuring a level of acetylated histone H3 (Ac-H3) in the midbrain DA precursor cells.

20. The method of claim 13, further comprising measuring a level of the transcription factors Nurr1, Foxa2 and Pitx3 in the midbrain DA precursor cells.
